(19) United States
(12) Patent Application Publication
(10) Pub. No.: US 2024/0374908 A1
(43) Pub. Date: Nov. 14, 2024

(54) SYSTEMS AND METHODS FOR NON-THERMAL PLASMA (NTP) MEDICAL TREATMENT

(71) Applicant: Mark G. FONTENOT, Lafayette, LA (US)

(72) Inventor: Mark G. FONTENOT, Lafayette, LA (US)

(21) Appl. No.: 17/716,847

(22) Filed: Apr. 8, 2022

Related U.S. Application Data
(63) Continuation-in-part of application No. 17/149,515, filed on Jan. 14, 2021, now Pat. No. 11,849,992.
(60) Provisional application No. 63/172,517, filed on Apr. 8, 2021.
(60) Provisional application No. 62/961,559, filed on Jan. 15, 2020.

(51) Int. Cl. A61N 1/44

(52) U.S. Cl. CPC A61N 1/44

(57) ABSTRACT

Embodiments include a system comprising a non-thermal plasma (NTP) device including a handpiece configured to generate NTP at a treatment site comprising an intraoral region. A sleeve is configured to removably couple to a distal region of the handpiece.

### RELATED APPLICATION

This application claims the benefit of United States (US) Patent Application No. 63/172,517, filed Apr. 8, 2021.

This application is a continuation in part of U.S. patent application Ser. No. 17/149,515, filed Jan. 14, 2021, which claims the benefit of U.S. Patent Application No. 62/961,559, filed Jan. 15, 2020.

### TECHNICAL FIELD

The present invention relates generally to systems and methods including the generation and application of non-thermal plasma.

### BACKGROUND

There is a need for systems and methods involving dielectric barrier discharge (DBD)-based non-thermal plasma (NTP) technology to cure disease, improve quality of life, and extend lives of patients.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE FIGURES

FIG. 1 is a block diagram of a DBD device configuration, under an embodiment.

FIGS. 2A-2D (collectively referred to herein as “FIG. 2”) show plasma creation using a volume DBD based device, under an embodiment.

FIG. 3 is a block diagram of a jet plasma system configuration, under an embodiment.

FIG. 4 shows a table including the component ingredients in an example embodiment of the SSGE, including water, perfluorodecalin, glycerin, phospholipids, Polawax, Vitamin E, and preservative, under an embodiment.

FIG. 5 shows a comparison of the pO₂ of hypoxic tissue, normal skin, ambient atmospheric oxygen, and SSOE, under an embodiment.

FIG. 6 shows an example of SSOE canister packaging, under an embodiment.

FIG. 7 is an example depiction of the SSOE including dissolved oxygen concentrated in high-solubility PFD droplets that are stabilized by emulsifying agents within an aqueous base, under an embodiment.

FIG. 8 includes a graph showing the oxygen outgassing of SSOE along with measurement of the oxygen partial pressure within the SSOE, which peaks at 817 mm Hg, under an embodiment.

FIG. 9 shows a bench testing configuration to measure the flow oxygen from SSOE across 1 mm of porcine skin, under an embodiment.

FIG. 10 shows the oxygen transfer data of the bench testing, including time versus transcutaneous partial pressure of O₂ (tcpO₂) across 1 mm of excised porcine skin, under an embodiment.

FIG. 11 shows a plot of time versus transcutaneous partial oxygen partial pressure (tcpO₂) of the control (post-face lift skin) and experimental (application of SSOE to post-face lift skin) on the contralateral side, under an embodiment.

FIG. 12 shows an example skin lesion presented for treatment, under an embodiment.

FIG. 13 shows the example skin lesion with SSGE topically applied over the lesion, under an embodiment.

FIG. 14 shows the example skin lesion following removal of the topical SSOE prior to the NTP treatment, under an embodiment.

FIG. 15 shows the example skin lesion during the NTP treatment, under an embodiment.

FIG. 16 shows the example skin lesion with at least a portion of SSOE remaining over the lesion for the NTP treatment, under an embodiment.

FIG. 17 shows a cylindrical fitting coupled at the distal end of the DBD handpiece and including SSOE topically placed on an interior wall of the cylindrical fitting, under an embodiment.

FIG. 18 shows the distal end of the DBD handpiece coupled to a cylindrical fitting including a port configured to couple to a gas supply or source, under an embodiment.

FIG. 19 is a block diagram showing controlled delivery of NTP, under an embodiment.

FIG. 20 includes tables showing effectiveness of NTP in eradicating microorganisms, under an embodiment.

FIG. 21 is a block diagram showing NTP-based onco-immunotherapy, under an embodiment.

FIG. 22 is an example floating electrode dielectric barrier discharge (FEDBD) NTP handpiece, under an embodiment.

FIG. 23 is another example FEDBD NTP handpiece, under an alternative embodiment.

FIG. 24 shows components of an NTP system including a controllable environment surrounding the distal end of the FEDBD handpiece and an enclosure coupled to the distal end of a sleeve surrounding the distal end of the FEDBD handpiece, under another alternative embodiment.

FIG. 25 includes tables showing effectiveness of NTP in eradicating microorganisms, under an embodiment.

FIG. 26 includes tables showing effectiveness of cold plasma in the inactivation of microbes, under an embodiment.

FIG. 27 is a block diagram showing NTP-based onco-immunotherapy, under an embodiment.

FIG. 28 shows an NTP system comprising a side-firing cold plasma configuration, under an embodiment.

FIG. 29 shows an NTP system comprising an end-firing cold plasma configuration, under an embodiment.

### DETAILED DESCRIPTION

### Non-Thermal (Cold) Plasma Used in Medicine

The development of devices to produce cold plasma under conditions of atmospheric pressure form the fundamental basis for the emergence of plasma-related application areas in medicine, and the medical literature describes the many clinical benefits of applications of non-thermal plasma in medicine. Cold plasma, also referred to as non-thermal plasma (NTP), comprises a myriad of active components such as charged particles, electric current, ultraviolet (UV) radiation, and reactive gas species which can function independently or synergistically. The combination of the different constituent ions and reactive species found in NTP have been shown to have positive clinical effects on various diseases, which renders NTP attractive for applications in medicine. For example, wound healing, antimicrobial effects on tissues, anti-inflammatory, tissue-stimulating, and treatment of benign, pre-malignant, and malignant conditions.

A variety of different NTP devices have been developed and tested for research purposes. In general, the NTP devices can be divided into two types: a) direct discharge in which plasma is created using a dielectric barrier discharge (DBD) device; and b) indirect discharge in which plasma is created distant from the plasma formation site and carried to a distant target site via a carrier (e.g., gas, helium, argon, nitrogen, etc.) (also referred to as jet plasma).

Devices that create and deliver NTP for medical use are currently available or under development. For example, kINPen MED (INP Greifswald/neoplas tools GmbH, Greifswald, Germany) is a jet plasma device that is created in a handpiece or similar housing with the plasma transport to the target tissue site via a gas such as argon. As a further example, PlasmaDerm® VU-2010 (CINOGY Technologies GmbH, Duderstadt, Germany) is a DBD-based device that generates NTP over the target tissue site.

The generation of NTP can be summarized as ionization and excitation of atoms or molecules of a neutral gas (e.g., argon, helium, oxygen, nitrogen, air, or mixtures thereof) via electron impact in response to supplying electrical energy; interaction of electrons and high energy states of atoms or molecules with reaction partners in the plasma phase and its vicinity (ambient air, liquids, surfaces), generating secondary and tertiary reactive species; and emission of electromagnetic radiation (UV, visible light, IR/heat, electric fields) formed by excitation and depletion processes or charge transport. Free electrons, high energy states of atoms and molecules along with ions and radicals in the plasma and those generated in secondary reactions are the main components of the chemical reactivity and biological activity of a plasma. The sum of the NTP derived chemical entities is often referred to as reactive species, for example, reactive oxygen species (ROS), reactive nitrogen species

(RNS), and combination of reactive oxygen and nitrogen species (RONS), all collectively referred to as plasma generated species (PGS). The NTP has been shown to kill and distress pre-cancer and cancer cells through biochemical pathways and immunogenic response, disrupt biofilms and eradicate bacteria, spores, viruses, fungi, including multidrug resistant organisms, and promote angiogenesis and tissue regeneration through stimulation of stem cells.

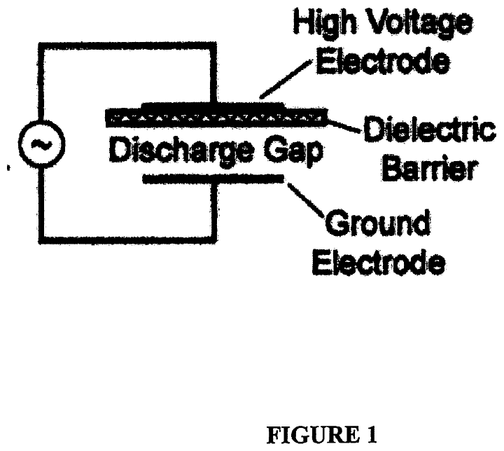

FIG. 1 is a block diagram of a DBD device configuration, under an embodiment. DBD configurations and principles to create NTP are characterized by the presence of an insulating material in the discharge path. Dielectric materials such as glass, quartz, and ceramics can be used, but embodiments are not so limited. Further, DBD devices that create plasmas can be created at atmospheric pressure but are not so limited. The operation of a plasma at normal (atmospheric) pressure with moderate high voltage amplitudes uses a discharge gap approximately in the range of 0.1-10 millimeters (mm). Because of the capacitive character of the discharge arrangement, alternating or pulsed high voltage is used. The high voltage amplitude is approximately in the range of 1 to 100 kilovolts (kV).

With reference to FIG. 1, a DBD is ignited by applying a high voltage between two electrodes, wherein at least one of the electrodes is insulated by a dielectric. By using the insulation, the occurrence of an arc discharge is prevented. Instead, many fine plasma filaments usually form between the electrodes, but they only have a very short lifetime in the range of nanoseconds.

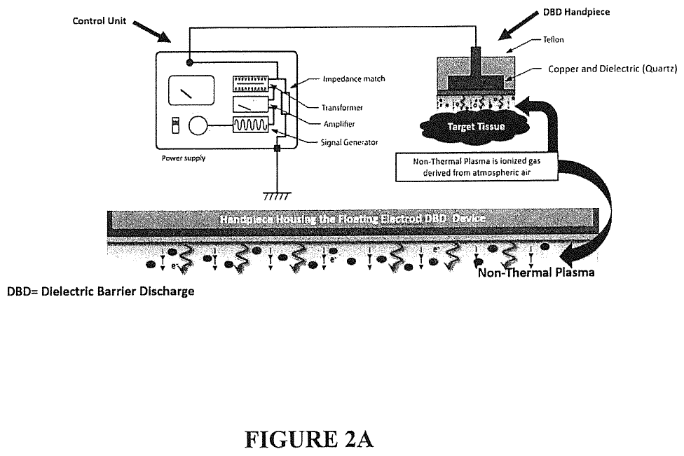

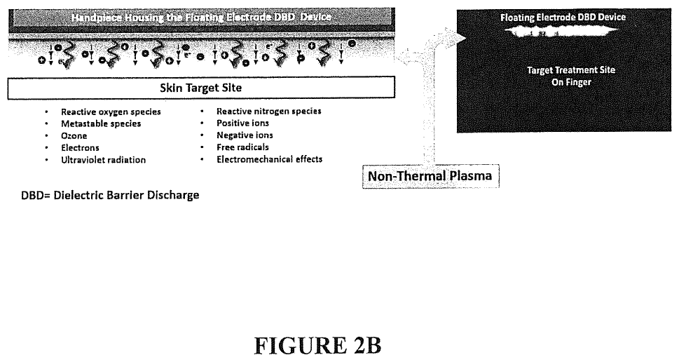

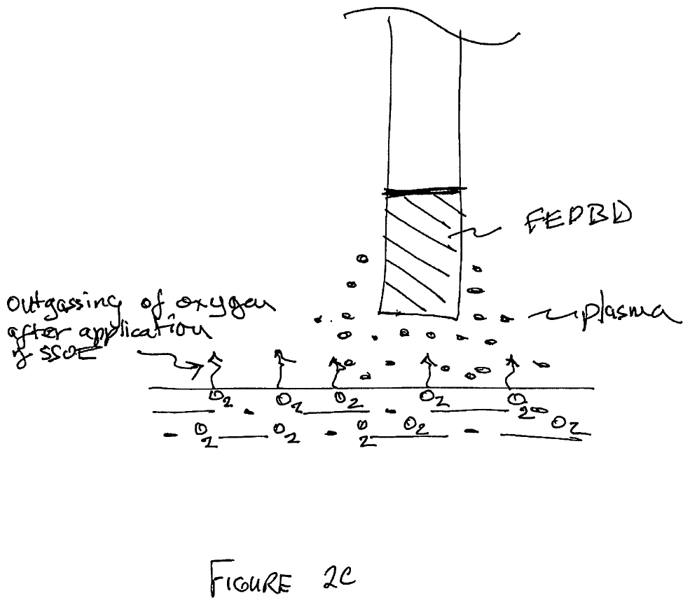

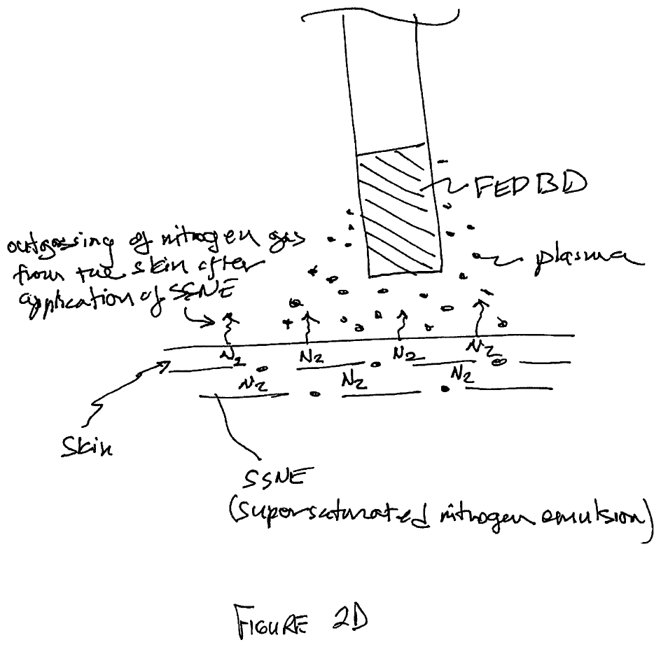

In volume DBD generated plasma, the plasma ignition occurs in a gap between an isolated high voltage electrode and the target to be treated. FIGS. 2A-2D (collectively referred to herein as “Figure 2”) show plasma creation using a volume DBD based device, under an embodiment. Consequently, cultured cells or living tissues in biomedical application are part of the discharge electrode configuration. The DBD device configuration in this example is referred to as floating electrode DBD. Plasma has direct contact with the target to be treated and the target is directly exposed to the electrical field that is necessary for plasma generation.

In surface DBD generated plasma, plasma is ignited around an individually designed electrode structure (e.g. circular or grid-like), which is isolated from a counter electrode. There is no direct contact of the active plasma with the target to be treated, instead impact is achieved by transport processes bringing the reactive species to the living tissue. With both DBD configurations, atmospheric air serves as the working gas for plasma generation. Both volume and surface DBD devices are suitable to generate plasmas over larger areas.

Embodiments of the devices described herein can be used with supersaturated gas emulsions applied to skin prior to treating the skin and any skin lesion with NTP. The supersaturated gas emulsions include but are not limited to supersaturated oxygen emulsion and supersaturated nitrogen emulsion. Subsequent outgassing of oxygen or nitrogen occurs after application of SSOE or SSNE to the treatment site, respectively. The skin is then treated using FEDBD NTP as described herein. FIG. 2C shows outgassing of oxygen after application of SSOE and treatment with FEDBD NTP, under an embodiment. FIG. 2D shows outgassing of nitrogen after application of SSNE and treatment with FEDBD NTP, under an embodiment. The NTP therapy is enhance and targeted with the application of SSNE, for example, creating reactive nitrogen species at the surface of the skin and within the cold plasma plum and associated plasma chemistry.

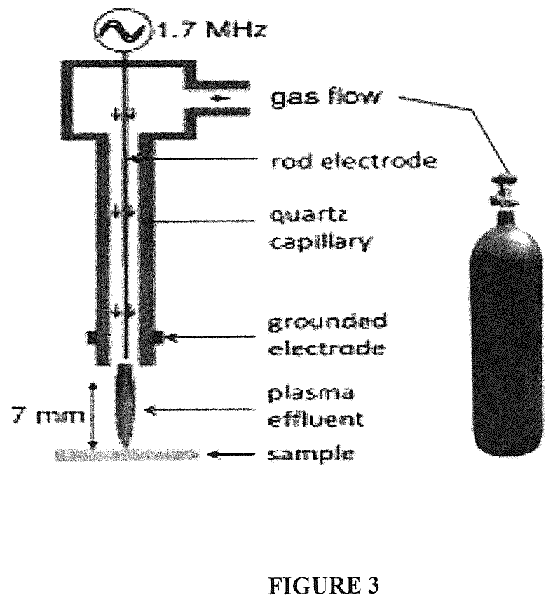

FIG. 3 is a block diagram of a jet plasma system configuration, under an embodiment. In the jet plasma device, the electrode is configured for plasma generation distant from the tissue target site and is generally located in or around a tube-like arrangement, in most cases inside a pen-like device. Diverse electrode configurations can be used, e.g. pin electrodes, ring electrodes, plate electrodes etc. The plasma is ignited inside the device using a working gas that is flowing through the tube. The so-called plasma effluent (or afterglow) is carried out along the gas flow and can be brought into direct contact with the target to be treated. In order to maintain a low temperature and to achieve excellent controllability of the discharge, most jet plasma devices use noble gases (e.g., helium or argon) as the working gas, often doped with small amounts of molecular gases (e.g., nitrogen, oxygen). The target to be treated is not part of the electrode configuration. However, because of the conductivity of the plasma and its afterglow, small electrical currents may pass to the target. By choosing an appropriate design of electrode and high voltage waveform these currents can be easily controlled.

An example of an argon-driven atmospheric pressure plasma jet device includes kINPen. A needle electrode inside a dielectric capillary is powered with a sinusoidal high voltage (2-6 kVₚₚ) with a frequency of 1.0-1.1 MHz (power less than 3.5 W in the hand-held unit). Argon gas with a flow rate of 3-5 standard liters per minute (slm) is used as the working gas. The plasma is generated at the tip of the needle and is subsequently released with the feed gas flow into the atmospheric environment, thereby generating a plasma effluent having a length of approximately 9-12 mm and a diameter of approximately 1 mm. Under these conditions, the electron density in the core plasma region near the high voltage electrode tip is on the order of 10¹²/cc and one order of magnitude lower in the visible effluent zone. However, electron density depends on several parameters and can be varied by admixture of molecular gases, such as oxygen and nitrogen.

The reactive species generated inside the plasma or as a result of plasma interactions with the surrounding media are considered the most important components responsible for biological plasma effects. In the kINPen, the argon-based plasma effluent is exposed to atmospheric air containing predominantly oxygen, nitrogen, and water. Traces of these, especially the water, are contained in the working gas in low ppm-amounts. These atmospheric air compounds are the precursors for secondarily generated non-radical and radical reactive oxygen and nitrogen species (ROS, RNS). Generation of ROS and RNS can also be modulated by controlled admixture of oxygen, nitrogen, water or air to the argon working gas flow, or by gas shielding and modification of the atmosphere around the plasma effluent. The effluent contains ROS, RNS, and residual high energy states target a liquid (a tissue). Current knowledge assumes that at the interface between gas phase and liquid (or solid) target as well as in the target bulk a considerable rearrangement of the ROS/RNS pattern occurs. Besides interactions of the plasma derived species among themselves, the interaction with target biomolecules results in the formation of diverse chemical structures.

Because all plasma sources for biomedical applications are working under atmospheric air conditions or use ambient air as working gas, the generation of ROS and RNS from air-based oxygen and nitrogen is a corresponding feature of all these plasma sources. However, the composition and quantity of plasma-generated ROS and RNS, as well as UV irradiance, electrical field and other characteristics, are strongly dependent on specific plasma sources and device parameters as working gas composition, power input and temperature.

The biological effects of NTP's are mainly based on ROS and RNS, which is primarily reasoned from experimental observations in vitro. Plasma effects on mammalian cells were found to be dependent on cell culture media composition, each exhibiting a different antioxidative potential. A multitude of investigations on plasma-liquid interactions has demonstrated the occurrence of ROS and RNS in liquid phases following plasma treatment. Moreover, it has been shown several times that liquids, such as water, physiological saline, or cell culture media become biologically effective following plasma treatment. This underlines a key role of liquid phase composition for biological plasma effects. ROS and RNS like superoxide (O2—·), hydrogen peroxide (H2O2), hydroxyl radical (·OH), singlet oxygen (1O2), ozone (O3), and RNS, such as nitric oxide (·NO), nitrogen dioxide (·NO2) and peroxynitrite (ONOO—), are transferred from plasma into the liquid environment of cells and tissue, or they are generated by a very complex network of secondary liquid reactions.

This insight of the central role of ROS and RNS has opened up the door to the field of redox biology to explain and interpret biological effects caused by NTP. Redox biology can be taken as the interface between the more or less unspecific impact of external factors and the specific response and adaptation of a cell or an organism via its metabolic and macromolecular structures. Meanwhile, it is well known that ROS and RNS are not solely harmful in cells, but also serve as signaling molecules via reversible oxidations and reductions of specific protein structures with cysteine as a major reaction target.

Depending on plasma treatment intensity and time, it is possible to inactivate mammalian cells by initializing programmed cell death in them. This is true particularly for cancer cells. After several reports on apoptosis induction in cancer cells in vitro, animal studies on transcutaneous plasma treatment of subcutaneously induced solid tumors could prove the general concept of plasma-supported tumor treatment. However, there are several open questions about the mechanisms of plasma attack on cancer cells, and possible selectivity with regard to healthy tissue or on possible secondary effects distant from the region of local plasma treatment. Most current hypotheses are based on a predominant role of plasma-generated redox active species. Briefly, it is assumed that NTP treatment causes apoptosis of cancer cells through a selective rise of intracellular ROS and corresponding ROS-based death pathways. In that regard, enhanced sensitivity of cancer cells may be caused by enhanced ROS levels in the cancer resulting from its unique metabolic activities.

Another hypothesis is based on the specific action of NTP via singlet oxygen generation and the subsequent induction of intercellular ROS-RNS-dependent apoptosis-inducing signaling. Plasma has been shown to induce proapoptotic effects more efficiently in tumor cells compared with the benign counterparts, leads to cellular senescence. In published in vivo clinical reports, plasma has been shown to reduce skin tumors. Plasma has also been shown to be effective in treating benign, pre-malignant, and cancerous skin conditions.

Embodiments described herein increase plasma generated species (PGS) over the target and in the target tissue, and include but are not limited to one or more of the following: hydrating the tissue target (for example skin) before plasma treatment; improving the gas permeability of the tissue target by applying a PFC and PFC emulsion with dissolved gases such as oxygen, nitrogen, and combinations of gasses thereof; increasing the partial pressure of select gasses such as nitrogen and oxygen in the target tissue by applying a PFC emulsion with dissolved select gasses to the target tissue; increasing the outgassing and number of select gasses at the atmosphere-tissue interface; and applying SSOE to skin and apply DBD plasma for example and create enhanced production of plasma generated species (PGS).

More particularly, an embodiment increases PGS by applying perfluorocarbon (PFC) emulsions containing high concentrations of dissolved gasses or combinations of dissolved gasses such as oxygen and/or nitrogen to penetrate tissues and increase the tissue concentration or partial pressure of delivered gas (ses). An alternative embodiment increases PGS by applying PFC topically to tissues to increase the solubility of plasma into the tissues. Another alternative embodiment increases PGS by applying PFC emulsions containing water to hydrate tissues.

### Supersaturated Gas Emulsion (SSGE)

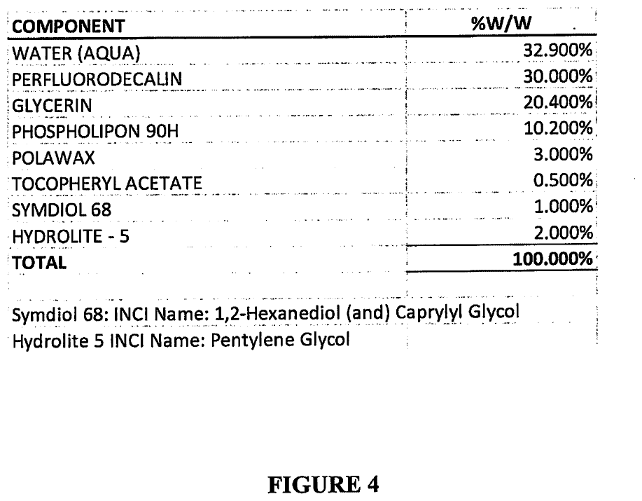

The SSGE comprises a dispersed phase of perfluorocarbon (PFC) droplets encapsulated within an aqueous continuous phase. Perfluorodecalin (PFD), a PFC, is selected for its high oxygen solubility, chemical inertness, and biocompatibility, but embodiments are not so limited. FIG. 4 shows a table including the component ingredients in an example embodiment of the SSGE, including water, perfluorodecalin, glycerin, phospholipids, Polawax, Vitamin E, and preservative, under an embodiment.

Gasses such as oxygen and nitrogen, which are not listed in this table, are added during the manufacturing process and after emulsification. The gas content of SSGE in the pressurized canister is dependent on the PFD concentration and the gas charging pressure used in the manufacture of the emulsion. The gas content of SSGE increases linearly with charging pressures. For example, if oxygen is used, the standard processing conditions for SSGE with oxygen as the gas calls for oxygenation at elevated pressures of at least 180 psig (charging pressure) of medical grade oxygen (created supersaturated oxygen emulsion or SSOE). The final equilibrium concentration of dissolved oxygen at 180 psig is approximately 1.8 ml O₂ (STP) per ml of emulsion. As a point of comparison, water at ambient conditions contains approximately 0.006 ml O₂ per ml water.

In this embodiment using oxygen, the SSOE is packaged under pressure to maintain the level of dissolved oxygen in solubilized form. The container pressure must be equal to or greater than the oxygenation pressure to prevent outgassing. When SSOE is dispensed, oxygen is outgassed from the emulsion and exposes a target tissue, for example skin, to oxygen.

In a current packaging configuration, SSOE, for example, comprises a pressurized canister containing 37 grams of SSOE. Each gram of SSOE includes approximately 1.8 ml of dissolved oxygen. Dispensing one gram of SSOE topically on the skin provides 1.8 ml of oxygen. Upon application, oxygen will diffuse from the SSOE into its ambient surroundings including the skin.

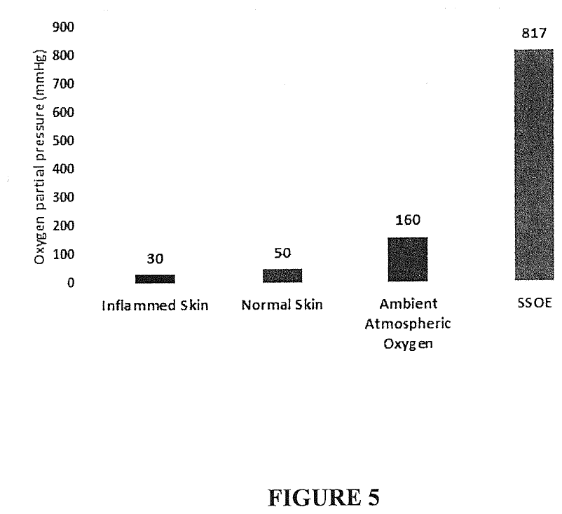

FIG. 5 shows a comparison of the pO₂ of hypoxic tissue, normal skin, ambient atmospheric oxygen, and SSOE, under an embodiment. In this comparison, the partial pressure of oxygen (pO₂) of air at one atmosphere (760 mmHg) is approximately 160 mmHg (21% oxygen). Normal pO₂ of arterial blood is approximately 100 mmHg. Normal tissue oxygen partial pressures range from 100 mmHg in pulmonary alveoli to 20 mmHg in liver parenchymal cells. Inflamed acute and chronic wounds can have oxygen tension levels (pO₂) between 0 and 30 mmHg even when surrounding subcutaneous pO₂ levels are on the order of 30 to 50 mmHg. Normal cell metabolism can be impaired at pO₂ levels of under 20 mmHg. FIG. 1 also shows the pO₂ of SSOE compared to other topical products and conditions.

### SSOE Pressurized Canister

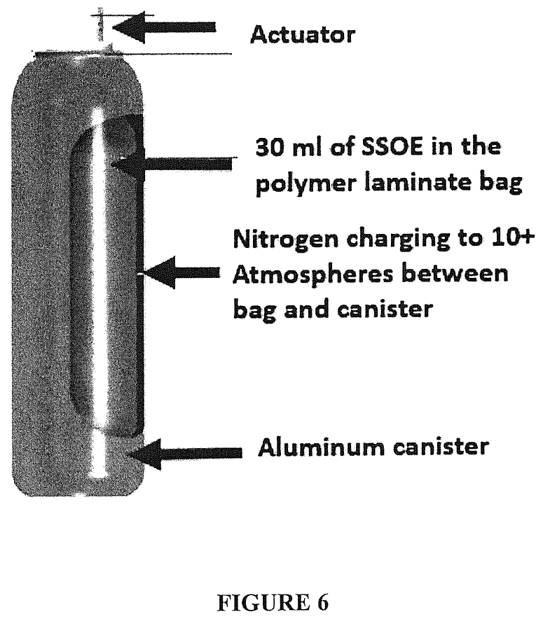

FIG. 6 shows an example of SSOE canister packaging, under an embodiment. In this configuration, SSOE is packaged in a pressured bladder canister including a one ounce volume (29.6 ml volume or 37 grams by weight). The bladder canister comprises an empty bladder with an internal canister pressure of 175±5 psig. During the manufacturing process, the SSOE is pumped into the bladder of the canister, resulting in an intra-canister pressure of approximately 230 psig. Since the canister is pressurized, the oxygen remains in the SSOE.

The SSOE is not an aerosol propelled product. Rather, a hermetically sealed bladder (pouch) inside the canister is filled with the SSOE while pressurized nitrogen gas surrounds this bladder. When the nozzle of the SSOE can is actuated, the gas in the canister squeezes the bladder and causes the SSOE to be discharged uniformly from the canister. All surfaces of the packaging that contact the SSOE either during its storage or delivery are non-reactive biocompatible materials. The bladder materials are FDA-compliant and keep the SSOE separated from the nitrogen gas propellant. The bladder consists of a laminated structure of polypropylene on the product contact side, a center layer of aluminum foil, and a PET (polyethylene terephthalate) outer layer.

### PFD Emulsion Formulation

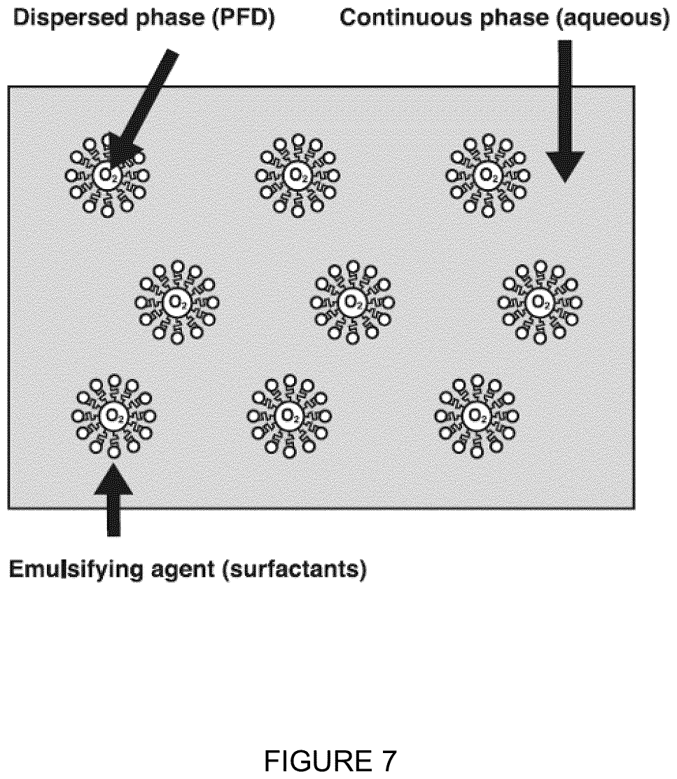

SSOE comprises a dispersed phase of PFD droplets encapsulated within an aqueous continuous phase. The chemical ingredients of the emulsion (e.g., see FIG. 4) include PFD, water, humectants, lubricity enhancers, and emulsion stabilizers (surfactants). PFD is chosen for its high oxygen solubility and chemical inertness. Mechanical agitation and homogenization of the heated ingredients forms a stable suspension of PFD microdroplets within the aqueous base. This emulsion is thermally and mechanically stable and will not separate into its constituent phases unless subjected to extreme conditions. FIG. 7 is an example depiction of the SSOE including dissolved oxygen concentrated in high-solubility PFD droplets that are stabilized by emulsifying agents within an aqueous base, under an embodiment. Small, discrete PFD droplets, shown in white, are depicted within the continuous aqueous phase, shown in blue. A surfactant shell surrounds each PFD droplet, encapsulating the dispersed PFD with a complex lamellar structure of surface-active stabilizers. These protective shells act to repel one another within the emulsion, preventing the microdroplets from coalescing and thereby imparting stability to the emulsion. Oxygen in the example of SSOE is added at high concentration during a secondary process after emulsification.

### Oxygen Content

The oxygen content of the packaged SSOE is determined primarily by PFD content (oxygen solubility of is approximately 10-20 times greater than water) and oxygen charging pressure during manufacture. The standard processing conditions for SSOE calls for oxygenation of at least 180 psig resulting in an equilibrium concentration of dissolved oxygen on the order of 1.8 ml O₂ (STP)/ml of SSOE, or three hundred (300) times the amount of oxygen contained in a glass of water.

The SSOE must be packaged under pressure for the dissolved oxygen to persist in solubilized form. The canister pressure must be equal to or greater than the oxygenation charging pressure to prevent outgassing of the oxygen from the emulsion.

### SSOE Bench Testing

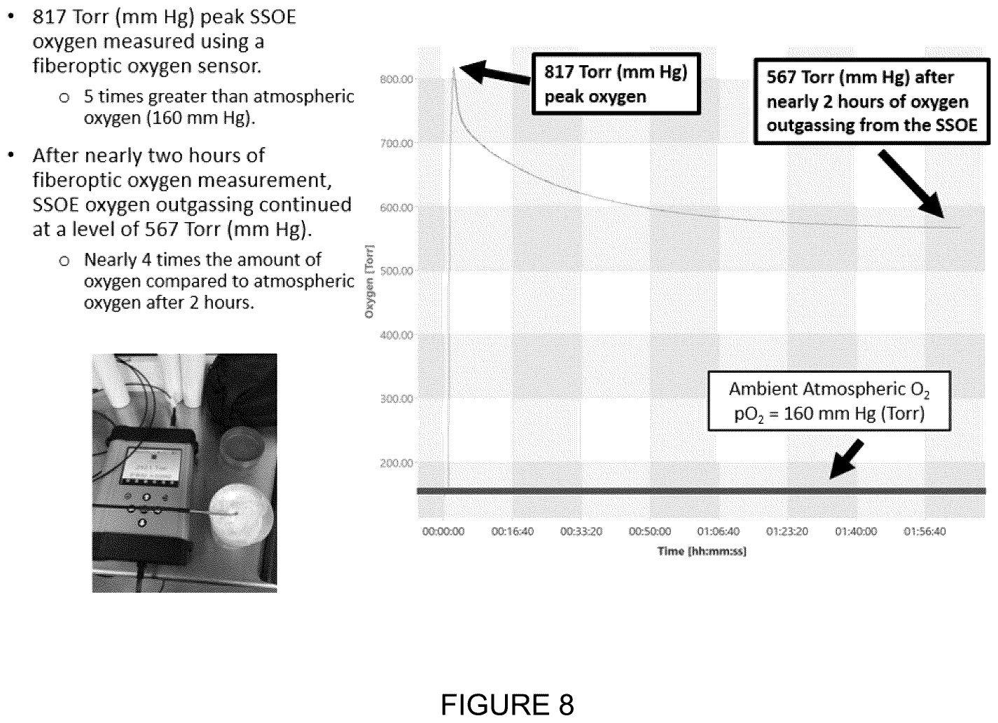

FIG. 8 includes a graph showing the oxygen outgassing of SSOE along with measurement of the oxygen partial pressure within the SSOE, which peaks at 817 mm Hg, under an embodiment. The oxygen partial pressure in this example was measured using a fiberoptic oxygen sensor. After nearly two hours, the oxygen outgassing continued as was found to be at 567 mmHg.

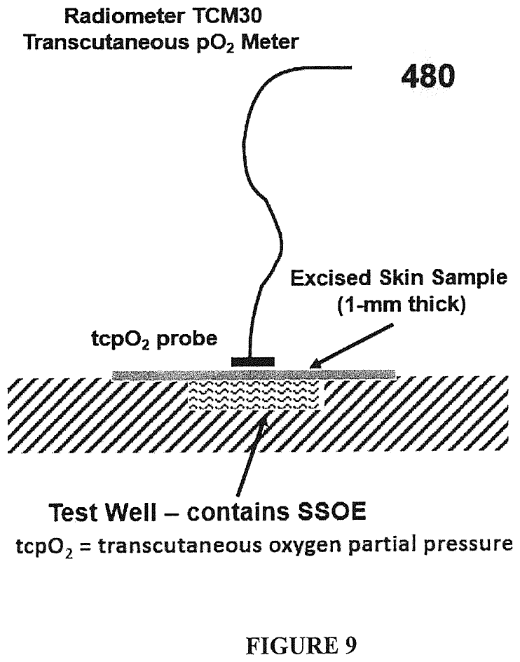

Bench testing was completed to measure the oxygen transfer from the SSOE to skin. FIG. 9 shows a bench testing configuration to measure the flow oxygen from SSOE across 1 mm of porcine skin, under an embodiment. In this example test, approximately 2.0 ml of SSOE was placed in a test well measuring 2.5×2.5×0.3 cm (depth). The test well was covered by a layer of epidermis of excised, non-viable porcine skin. The epidermis and underlying dermis is trimmed to a uniform thickness of approximately 1 mm over the area of the test well. A transcutaneous pO₂ probe was placed on the porcine skin over the test well containing the emulsion. The oxygen concentration in the tissue was measured over time with a standard transcutaneous pO₂ probe (Radiometer TCM) applied to the skin surface opposite the applied emulsion. The skin tissue pO₂ is equilibrated with ambient surroundings at time zero and rises over time as the diffusing oxygen from the emulsion penetrates through the porcine skin.

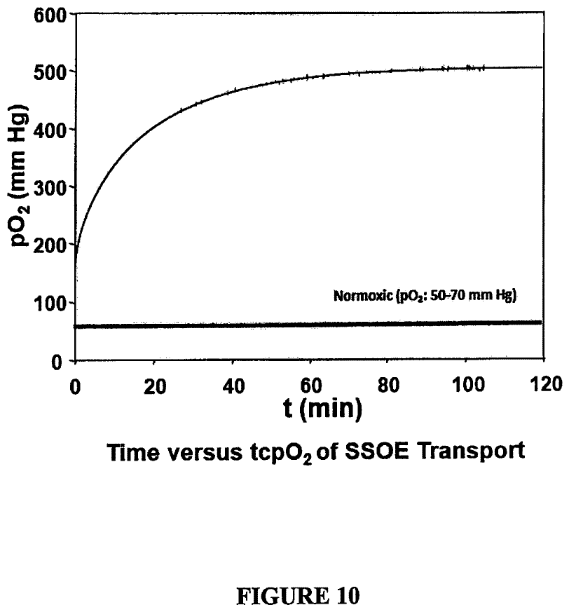

FIG. 10 shows the oxygen transfer data of the bench testing, including time versus transcutaneous partial pressure of O₂ (tcpO₂) across 1 mm of excised porcine skin, under an embodiment. Oxygen transfer from the SSOE occurs via diffusion (concentration gradient) and convection (mass transfer by absorption). The tissue oxygenation level, or pO₂, is monitored after the SSOE is applied at time zero. After a few minutes, a sharp increase in oxygenation is observed from ambient conditions to an elevated, sustained peak value approaching 500 mmHg. Because the skin sample is excised and non-absorbent, the oxygenation level can be sustained for many hours after only one SSOE application.

On skin samples greater than twice this thickness (1 mm), SSOE has demonstrated that the oxygenated emulsion penetrates the tissue and elevates pO₂ levels for a sustained period. These bench tests coupled with clinical measurements of transcutaneous partial pressure of oxygen (tcpO₂) after application of SSOE and combined with other corollary data, show proof that the technical goal of delivering and achieving a local hyperbaric environment.

### SSOE Oxygen Transfer

Because the partial pressure of O₂ in the SSOE is greater than that in atmospheric air, a gradient exists between the SSOE and the atmosphere. Once dispensed from the canister, oxygen slowly begins to move out of the PFD emulsion, through the phospholipid membrane, and through the water of the SSOE down the gradient via the process of diffusion. The oxygen preferentially leaves the SSOE through surfaces of the emulsion in contact with tissue or air. Because of the slow nature of the diffusion process, release of oxygen from the SSOE is very gradual. A layer of SSOE approximately 3 millimeters is capable of delivering oxygen to a contacting tissue surface at levels above those found in arterial blood for at least several hours. For example, in the treatment of burn, if 2 ml of SSOE is topically applied, approximately 4 to 5 ml of oxygen is available, much of which is delivered to the covered tissue and over a period of at least an hour.

The SSOE is a biocompatible emulsion that creates a local hyperoxic environment in skin. The SSOE absorbs readily into the skin for maximum oxygen delivery. After topical application, skin interstitial pO₂ increases 3-to 5-fold from 20 to 35 mm Hg pO₂ pre-application to 100 mm Hg pO₂ post-application (FIG. 10)

Oxygen transfer from the SSOE into the skin occurs via diffusion and convection. Diffusion describes the molecular migration of oxygen from the SSOE into the skin and is proportional to the large oxygen concentration gradient between the applied emulsion and skin tissues. Duration of effect over hours as been observed in vitro, due to the characteristically slow diffusion process. A second important oxygen transfer process occurs via convection, wherein the SSOE is absorbed directly into the skin and transports the solubilized oxygen directly to the tissues. Rapid absorption of the SSOE, which occurs on a time scale much faster than diffusion, ensures efficient delivery of the SSOE's oxygen load to the application area.

The combination of diffusive and convective transfer maximizes the effective dose of oxygen to the skin while minimizing the amount of oxygen lost to the ambient surroundings during dispensation and delivery. The SSOE formulation can be modified for rapid or slow, sustained absorption depending upon the desired rate of oxygen transfer.

### SSOE Clinical Measurements

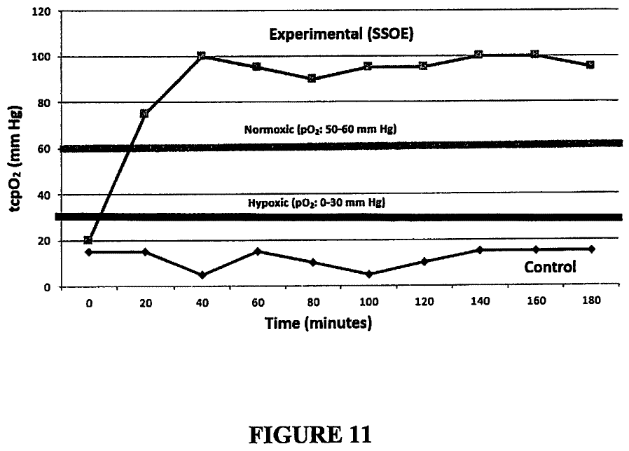

FIG. 11 shows a plot of time versus transcutaneous partial oxygen partial pressure (tcpO₂) of the control (post-face lift skin) and experimental (application of SSOE to post-face lift skin) on the contralateral side, under an embodiment. The tcpO₂ increases five-fold from approximately 20 mmHg tcpO₂ (control) to 100 mmHg tcpO₂ after the application of SSOE to immediate post-face lift skin (experimental). The data in this plot represents the decrease in skin oxygen tension following facelift surgery. Oxygen tension was measured at 15mmHg, which is a much lower when compared to pre-operative measure levels of 60 mmHg. The subsequent 5-fold increase following the application of SSOE shows oxygen transfer from SSOE to skin.

The cutaneous uptake of atmospheric oxygen contributes significantly to the oxygen supply of human dermis and epidermis. The upper skin layers to the depth of 250 μm to 400μm are almost exclusively supplied by external oxygen, where the oxygen transport of the blood has a minor influence. SSOE delivers over 800+ mmHg of oxygen topically to skin, which is a 20+ fold increase in oxygen compared to hypoxic skin such as chronic wounds or injured skin.

### SSGE In Combination with NTP

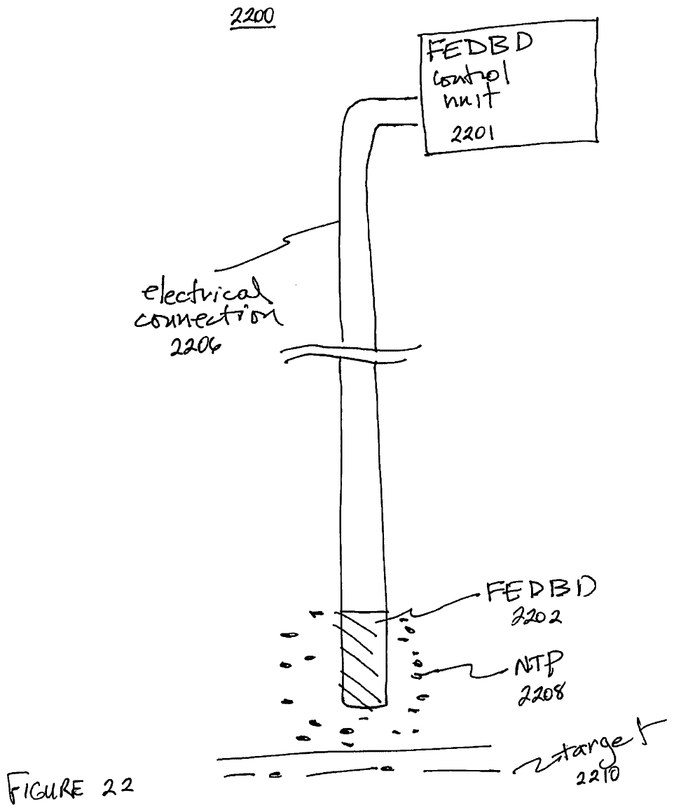

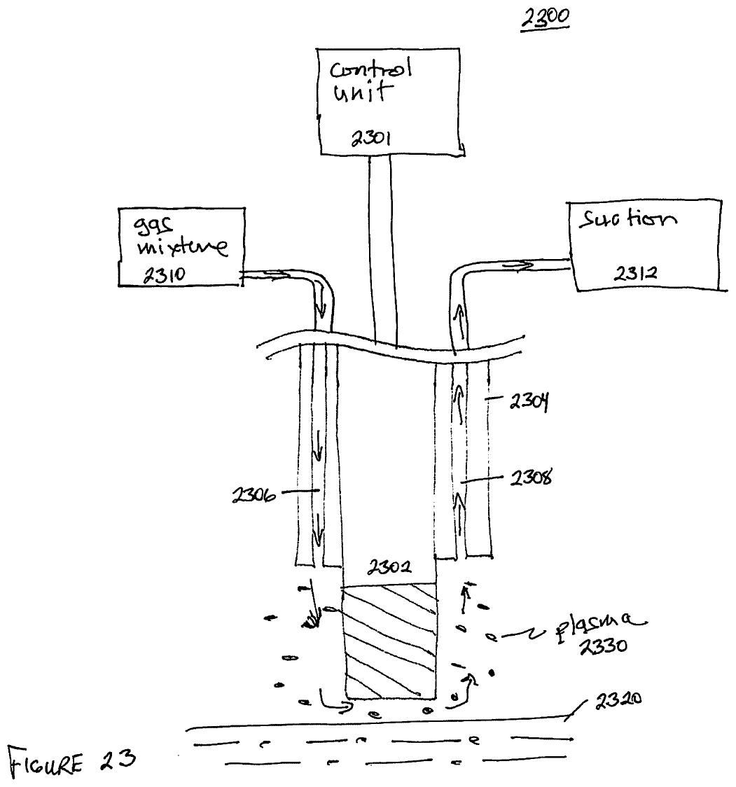

Embodiments described herein use NTP with SSGE to generate a cloud of RONS over target tissue and facilitate transport of RONS across cell membranes to disrupt internal cellular function and biochemical pathways, leading to cell death (apoptosis). The NTP system comprises a control unit comprising a power supply, control panel, and software. A DBD handpiece couples to the control unit and houses or carries the DBD electrode as described in detail herein. The DBD handpiece includes two configurations including a pencil-shaped configuration to treat single lesions (see FIG. 23), and a cylindrical-shaped configuration to treat large lesions (field treatment) (see FIG. 22). The NTP system of embodiments also includes a consumable comprising a disposable, single-use polymer endcap for the handpiece. The handpiece of the NTP system creates NTP at the target site under conditions of room temperature and ambient atmosphere, and results in no thermal tissue damage. Treatment sites include sites with small and large surface areas, and treatment times are approximately one to two minutes per treatment site.

Examples are now presented showing various SSGE embodiments in combination with NTP.

### Topical SSGE Application on a Lesion before NTP Treatment

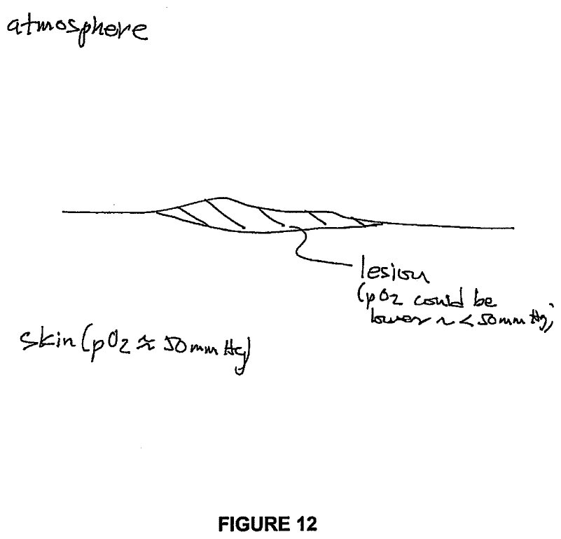

An example includes treatment of a skin lesion such as actinic keratosis or skin cancer using NTP. FIG. 12 shows an example skin lesion presented for treatment, under an embodiment.

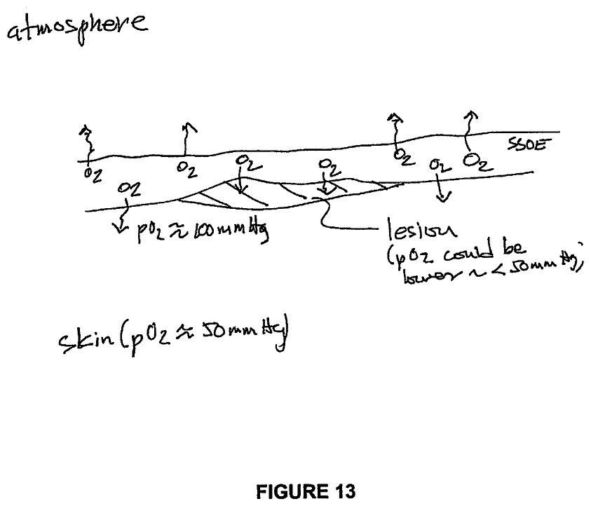

Before NTP treatment, SSGE is topically applied over the skin lesion and the area to be treatment. FIG. 13 shows the example skin lesion with SSGE topically applied over the lesion, under an embodiment. In this example, SSOE is applied to increase the presence of oxygen within the skin and immediately over the skin at the atmosphere-skin interface. The SSOE is applied to the lesion for seconds to minutes. If the physician elects to increase the presence of nitrogen, a supersaturated nitrogen emulsion (SSNE) would be applied. If a combination of oxygen and nitrogen are desired, then a desired mixture of nitrogen and oxygen gas would be used in the manufacture of the SSGE resulting in a supersaturated nitrogen-oxygen emulsion (SSNOE) NTP treatment.

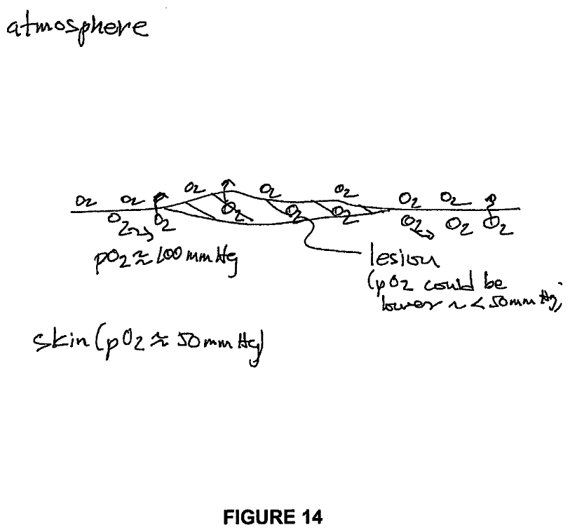

Immediately before NTP treatment, the SSOE can be wiped away with NTP treatment to immediately follow. FIG. 14 shows the example skin lesion following removal of the topical SSOE prior to the NTP treatment, under an embodiment.

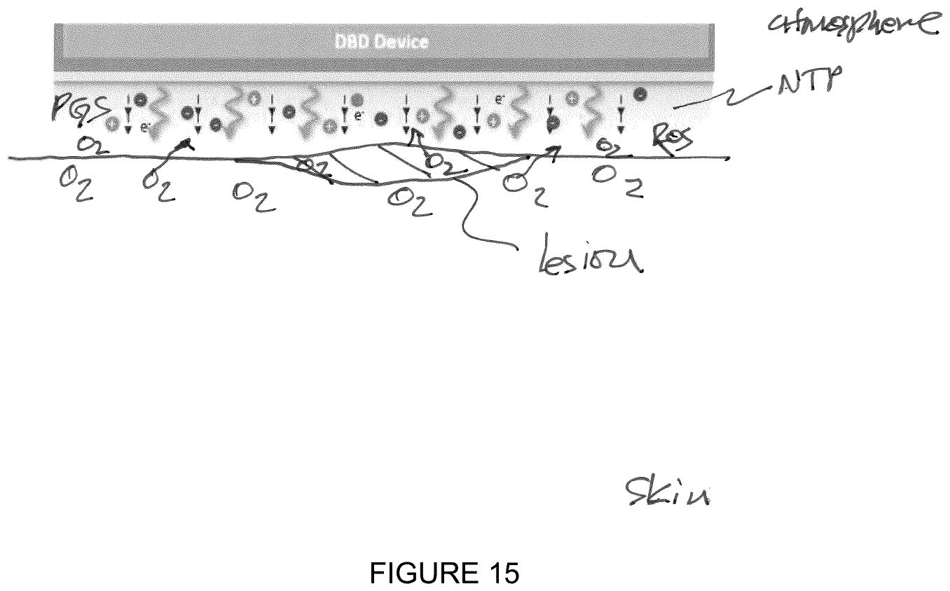

FIG. 15 shows the example skin lesion during the NTP treatment, under an embodiment. The increased oxygen during NTP treatment increases the concentration of ROS. In addition, the PFD based SSOE is absorbed and adsorbed into the skin, wherein the PFD increases permeability and solubility of oxygen across the skin including ROS and other PGS. Furthermore, high water content of the SSOE increases the hydration of the skin, which improves the flow of PGS into the skin and lesion.

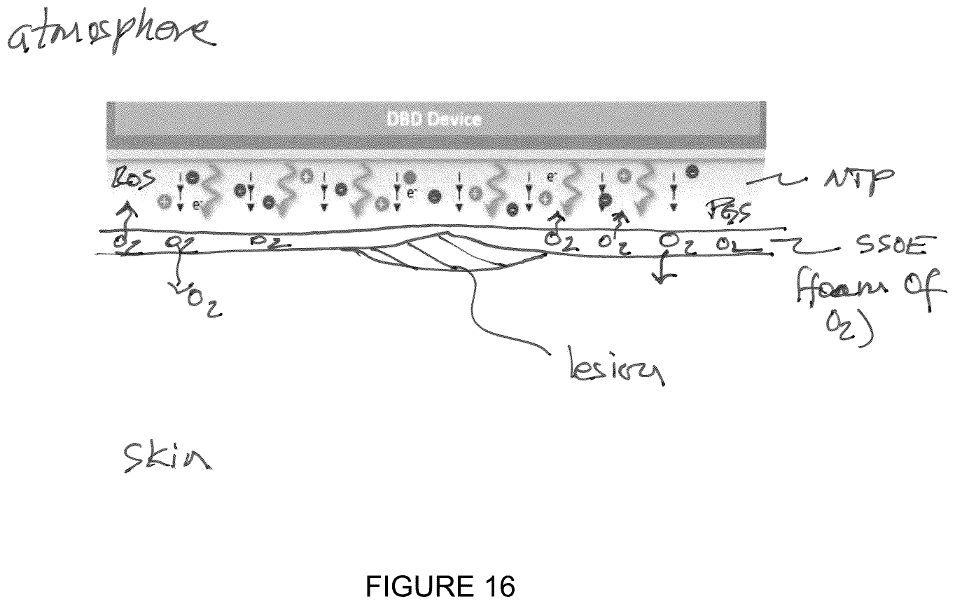

Alternatively, instead of wiping away the SSOE prior to the NTP treatment, the SSOE can be left in place or slightly removed leaving a thin layer or film of SSOE over the lesion. FIG. 16 shows the example skin lesion with at least a portion of SSOE remaining over the lesion for the NTP treatment, under an embodiment. As the oxygen outgasses from the SSOE, the SSOE expands into a porous, supersaturated oxygen foam over the lesion. The application of the NTP treatment over the thin film of SSOE covering the lesion increases the concentration of ROS and other PGS in the foam and over the lesion, allowing for improved ROS and PGS availability and delivery.

### SSGE Application to Interior Wall of DBD Handpiece Cylindrical Fitting

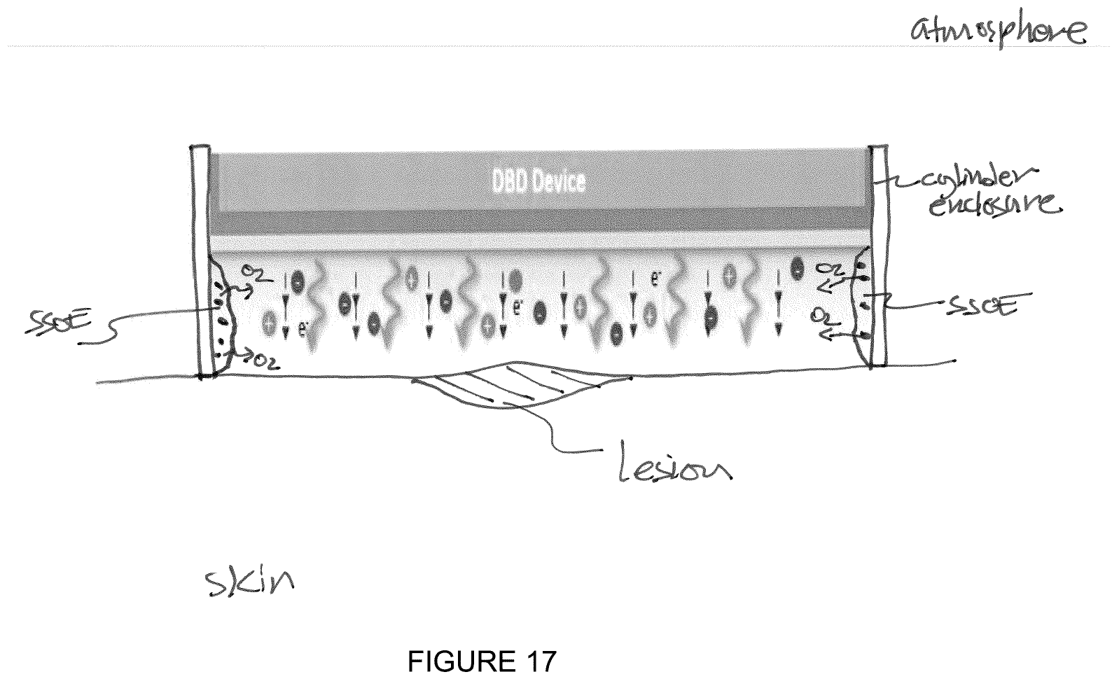

In the delivery of DBD NTP, the distance between the distal end of the DBD handpiece and the skin or target site should be kept at a relatively constant distance. Specifically, in an example of a cylindrical DBD handpiece, a reversible coupled cylindrical fitting can be placed at the distal end of the DBD handpiece to provide a desired and fixed distance between the skin and the distal end of the DBD handpiece. In an embodiment, the SSOE can be topically placed on the interior wall of the cylindrical fitting. The SSOE outgasses oxygen in the area or zone, increasing the oxygen concentration and increasing the ROS and PGS when NTP is created. FIG. 17 shows a cylindrical fitting coupled at the distal end of the DBD handpiece and including SSOE topically placed on an interior wall of the cylindrical fitting, under an embodiment.

### Controlling the Atmosphere in the NTP Zone in a DBD Created NTP Device

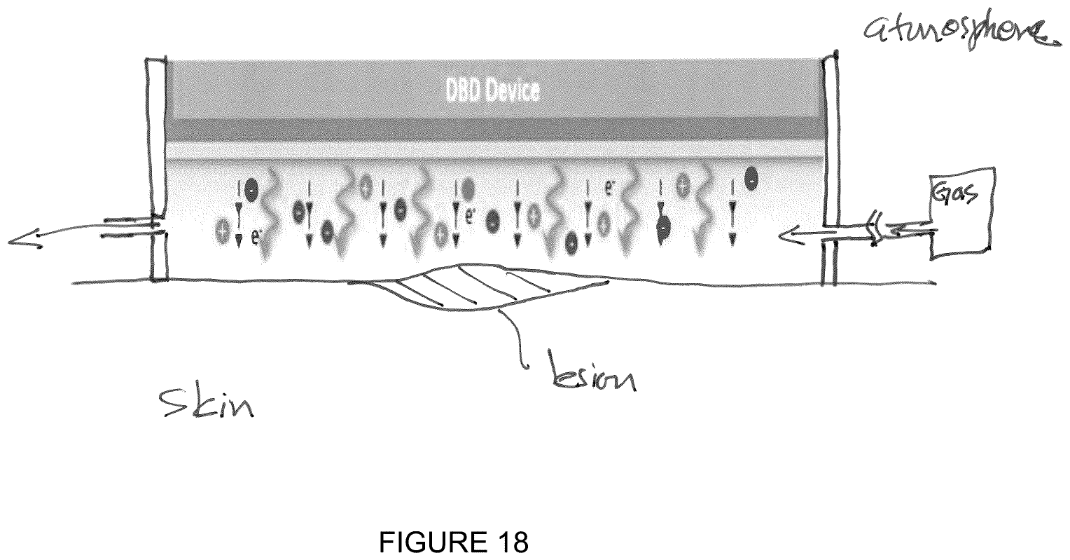

The atmospheric constituents in the volume created between the distal end of a DBD handpiece and the skin can dictate the reactive species and PGS in general. In an embodiment, the cylindrical fitting includes a port configured to couple to a gas supply or source. FIG. 18 shows the distal end of the DBD handpiece coupled to a cylindrical fitting including a port configured to couple to a gas supply or source, under an embodiment. The port is configured to allow gas to flow from a coupled gas source into the volume between the distal end of the DBD handpiece and the skin. The gas can be selected to create the desired PGS, e.g., oxygen, nitrogen, or combination of oxygen and nitrogen.

### Indirect Delivery of PGS Using SSGE

The SSGE can be blended with, for example, Ringer's lactate solution (RLS). In one embodiment, RLS is blended into the PFD emulsion, then oxygen is added, resulting in RLS-based SSOE. The RLS-based SSOE can be exposed to the NTP for a period of time, then delivered topically to the target tissue site.

### Use of SSOE in Conjunction with Superficial Radiation Therapy

Hyperbaric oxygen (HBO) therapy (HBOT) has been used in conjunction with radiotherapy (RT) and chemotherapy (CT) for the treatment of cancer. HBOT and the systemic delivery of oxygen has been shown to work synergistically with RT and CT in the treatment of cancer.

RT uses the so-called classical oxygen effect in tumor treatment. Upon exposure to radiation, water molecules undergo radiolysis to form unstable hydrogen and hydroxyl radicals. Hydrogen radicals react with molecular oxygen, yielding unstable perhydroxyl radicals and hydrogen peroxide, which cause serious DNA strand damage and consequently lead to cell death. Thus, radiation treatment gives an optimal therapeutic result in well-oxygenated tumor tissue. It was observed that mice breathing pure 1 atm oxygen required a one-third smaller dose of X-rays than mice that were breathing air to achieve similar cancer regression. HBO might play two possible roles when combined with RT, namely it may act as a radiosensitizer, which enhances the effect of radiation, or it may act as a therapeutic agent, reducing delayed radiation injury. A combination of HBO and radiotherapy reduces tumor growth and improves local tumor control, resulting in increased survival time.

Superficial radiation therapy (SRT) is used to treat skin cancers. SRT deliver radiation to the skin. To enhance SRT, SSOE would be applied to the lesion to be treated before treatment. The SSOE is left over the lesion for 1 to 30 minutes, then gently removed or can be left in-place during SRT. The lesion is then subjected to SRT. The SSOE-SRT combination is repeated until SRT is completed. The SSOE can also be used between SRT session and post-SRT to reduce radiation dermatitis and skin side effects of SRT.

### FEDBD NTP and Plasma Chemistry Technology

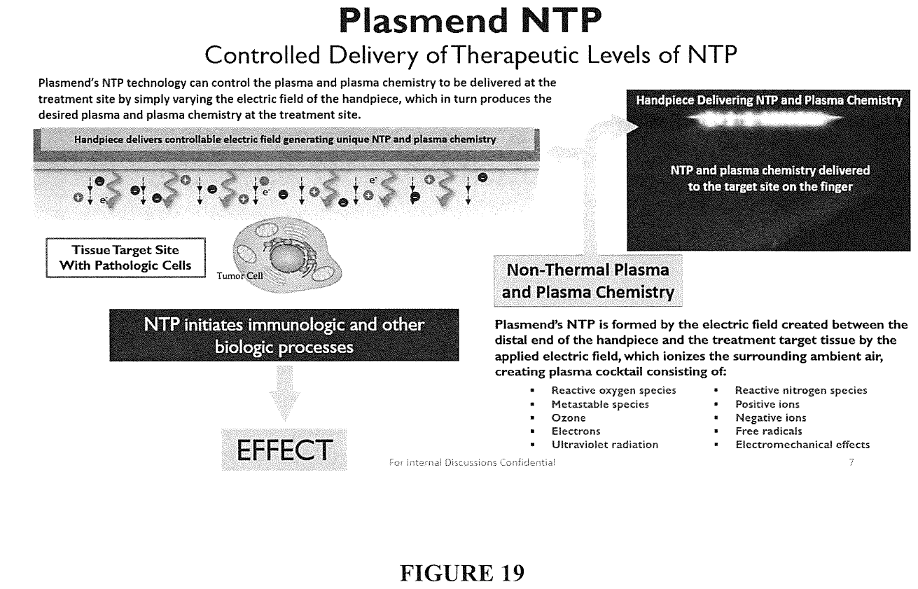

FIG. 19 is a block diagram showing controlled delivery of NTP, under an embodiment. The NTP is generated over the target tissue site and comprises excited gas molecules, positively and negatively charged ions, free electrons, reactive nitrogen species (RNS), reactive oxygen species (ROS), free radicals, and molecule fragments. The NTP platform can vary the electric field to produce a unique plasma cocktail consisting of plasma chemistry having the same reactive oxygen and nitrogen species (RONS) as cells. The NTP cold plasma technology of embodiments is a highly controllable redox stress delivery system, resulting in plasma immunotherapy. The NTP has been shown to kill and distress pre-cancer and cancer cells through biochemical pathways and immunogenic response, disrupt biofilms and eradicates bacteria, spores, viruses, fungi, including multidrug resistant organisms, promote angiogenesis and tissue regeneration through stimulation of stem cells, and/or create nano-streamers in the presence of plasma and plasma chemistry that strikes, penetrate, and controllable ablate skin cellular layers, resulting in skin rejuvenation.

The electrical parameters of the control unit are set by the user (provider), which powers the FEDBD NTP handpiece, creating non-thermal plasma and associated plasma chemistry at the implant surface. The plasma and plasma chemistry is generated at room temperature under ambient atmospheric conditions. Embodiments of the NTP device configurations include power settings with corresponding handpiece configurations to address a number of clinical applications. In particular, the FEDBD creates and electric field and resulting plasma and plasma chemistry consisting of at least reactive oxygen species, reactive nitrogen species, metastable species, ozone, electrons, and ultraviolet radiation. The implant is plasma treated for up to approximately 10 minutes. Exposing the implant to the FEDBD plasma and plasma chemistry removes any residual organics resulting in a super-clean sterilized implant surface with optimized surface energy and wettability immediately prior to placement.

Upon completion of the plasma and plasma chemistry surface treatment at the time of surgery, the implant has improved wettability and surface energy, which is surgically placed.

In the case of treating a tooth surface prior to the placement of dental material such as bonding a veneer with a resin, cementing a crown, and placement of esthetic resin, the prepared tooth is treated using the FEDBD handpiece for up to approximately one (1) minute. After treatment of the tooth structure, any biofilm is removed and the tooth surface energy and wettability are improved, yielding optimal tooth surface properties to improved dental material performance, e.g., resin bonding.

Non-thermal plasma (NTP) or cold plasma and associated plasma chemistry as referenced herein is generated at room temperature by the input electrical current to a floating electrode dielectric barrier discharge (FEDBD) handpiece in the presence of the atmosphere (78% nitrogen gas and 21% oxygen gas), which in turn generates a NTP (or cold plasma) and plasma chemistry consisting of ionized atoms and molecules. More specifically, NTP and the associated plasma chemistry is created at room temperature and in ambient air, and is comprised of a reactive mix of electrons, ions, excited atoms and molecules referred to as reactive species such as reactive oxygen species and reactive nitrogen species as well as UV light and other frequencies.

NTP technology can control the plasma and plasma chemistry to be delivered at the treatment site by varying the electric field of the handpiece, which in turn produces the desired plasma and plasma chemistry at the treatment site. NTP is formed by the electric field created between the distal end of the FEBDB handpiece and the treatment target tissue by the applied electric field, which ionizes the surrounding ambient air, creating plasma cocktail consisting of reactive oxygen species, metastable species, ozone, electrons, ultraviolet radiation, reactive nitrogen species, positive ions, negative ions, free radicals, and electromechanical effects.

NTP or cold plasma and associated plasma chemistry has been shown to kill and distress pre-cancer and cancer cells through biochemical pathways and immunogenic response, and to disrupt biofilms and eradicate bacteria, spores, viruses, fungi, including multidrug resistant organisms. NTP or cold plasma and associated plasma chemistry has also been shown to promote angiogenesis and tissue regeneration through stimulation of stem cells, and to create nano-streamers in the presence of plasma and plasma chemistry that strikes, penetrate, and controllable ablate skin cellular layers, resulting in skin rejuvenation.

As described herein, NTP or cold plasma and plasma chemistry is created over the target tissue site and comprises excited gas molecules, positively and negatively charged ions, free electrons, reactive nitrogen species (RNS), reactive oxygen species (ROS), free radicals, and molecule fragments. The NTP system of embodiments can vary the electric field to produce a unique plasma cocktail consisting of plasma chemistry having the same reactive oxygen and nitrogen species (RONS) as cells. The plasma technology of the NTP system of embodiments therefore enables a highly controllable redox stress delivery system, resulting in plasma immunotherapy.

When considering application to procedures involving dental implants, the electrical parameters of the NTP control unit of embodiments, which powers the FEDBD handpiece, can be established and set by the user and result in creation of non-thermal plasma and associated plasma chemistry at the surface of the dental implant. The plasma and plasma chemistry are generated at room temperature under ambient atmospheric conditions. Various NTP device design configurations described herein incorporate power settings with corresponding FEDBD handpiece configurations to address a number of clinical applications. In particular, in the presence of ambient air, the FEDBD creates an electric field and associated plasma and plasma chemistry comprising at least reactive oxygen species, reactive nitrogen species, metastable species, ozone, electrons, and ultraviolet radiation. The implant is plasma treated for up to 30 minutes, for example. Exposing the implant to the FEDBD plasma and plasma chemistry removes residual organics resulting in a super-clean implant surface with optimized surface energy and wettability immediately prior to implant placement. Upon completion of the plasma and plasma chemistry surface treatment at the time of surgery, the implant has improved wettability and surface energy, which is then surgically placed.

### Improving Wettability of Implants

Implants, when in contact with the biological environment, are characterized by dynamic changes in their surface properties. Interaction between tissues and the implant surfaces begin immediately after the placement. For bone implants, such as dental implants, the cascade of reactions that occur between the biological environment and implant surface, leads to the formation of a biofilm, which modulates the host's cellular responses. This event involves the blood clotting and the implant, with a thin layer of serum protein progressing to granulation tissue, followed by immature woven bone. The bone formation begins early, during the first week, through the promotion of osteoblast differentiation, production of osteogenic factors, cytokines and growth factors. The primary bone that includes trabecular of woven bone is substituted by parallel fibered and/or lamellar bone and marrow. Between 1 and 2 weeks, the bone tissue responsible for primary mechanical stability of the implant, immediately lateral to the implant region, is resorbed and substituted by newly formed bone.

The surface properties of dental implants are one of the key parameters affecting the speed of osseointegration. Surface energy and hydrophilicity (or wettability) are important features of an implant when considering implant-tissue interface upon placement. In particular, the implant surface energy for bone implants is an important factor for regulating osteogenesis. Hydrophilicity or wettability is a property that affects the osseointegration of dental implants. Hydrophilicity presents major advantages during the initial stages of wound healing and during the cascade of events that occurs during osseointegration, facilitating bone integration. Many animal and clinical studies support the positive effect of hydrophilicity on osseointegration by significant improvements in bone-implant contact (BIC) and bone anchorage during the early stages of bone healing.

In summary, wettability and surface energy are important attributes when considering the tissue-implant interface or, in the case of placing dental materials on or in teeth, the interface between dental materials (resins, cements, etc.) and tooth structure. Improving the wettability and surface energy of tooth structure to receive, for example, bonding materials improves the bonding material-tooth interface for improved performance.

Embodiments described herein include the use of NTP generated via a FEDBD coupled to an electrical control unit to create cold plasma and plasma chemistry in the presence of ambient air to treat the surfaces of implants to improve surface energy and wettability and eventually implant-tissue interface, and treat teeth that have been prepared to receive a biomaterial such as cement when cementing a crown (treat the crown preparation before cementation) or placing veneers (treat the prepared tooth surface before bonding the veneer) for improved bonding and biomechanical performance.

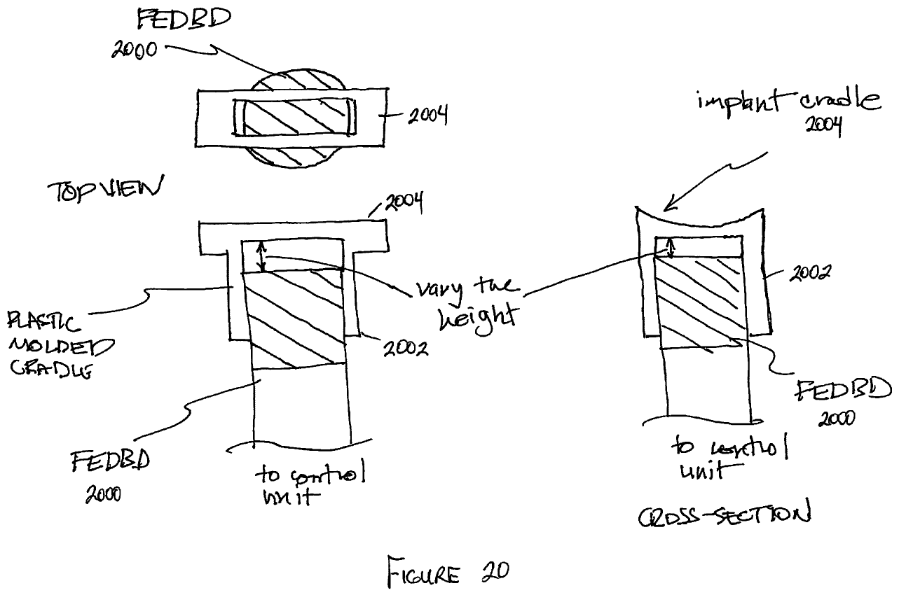

In embodiments, treatment of surfaces of implants comprises two approaches including the use of two respective variations of DBD or FEDBD devices, also referred to herein as dental handpieces. FIG. 20 is an example DBD or FEDBD NTP handpiece 2000 comprising a cylindrical-shaped configuration for use in treating large lesions, under an embodiment. The handpiece 2000 comprising the cylindrical-shaped configuration includes a distal end having a cylindrical shape configured to be optionally fitted with a removably coupled disposable polymer spacer 2002. The spacer 2002 of an embodiment is configured to slide over the distal end of the handpiece 2000 such that the distance between the distal end of the handpiece 2000 and a cradle or fixture 2004 of the spacer 2002 is variable.

The cradle 2004 is configured to hold and position the implant adjacent to the FEDBD. The cradle, which can be fabricated from a plastic injection mold, can be adjusted such that the implant can be position adjacent to the FEDBD as appropriate to the corresponding dental procedure (e.g., 1 mm gap from the distal end of the FEDBD handpiece to the surface of the implant, etc.). Thus, the variable configuration of the cradle 2004 relative to the distal end of the handpiece 2000 enables the user to select a desired device-tissue gap appropriate to the type of treatment being delivered. Using the FEDBD handpiece 2000. In the treatment approach using this device, when the FEDBD handpiece 2000 is powered to an operational state, cold plasma and associated plasma chemistry is created at and adjacent to the surface of the implant, exposing the implant surface to cold plasma and associated plasma chemistry.

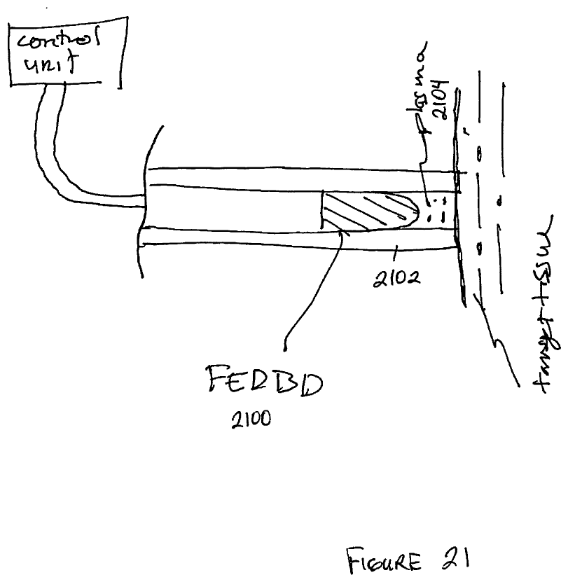

In an alternative approach, the implant is held by a user and the surface of the implant is treated using an FEDBD handpiece 2100 coupled to a control unit. FIG. 21 is an alternative example DBD or FEDBD NTP handpiece 2100 comprising a pencil-shaped configuration for use in treating single lesions, under an alternative embodiment. The handpiece 2100 comprising the pencil-shaped configuration includes a distal end configured to be optionally fitted with a removably coupled disposable polymer spacer 2102. The spacer 2102 of an embodiment, which is provided in numerous different lengths, is configured to slide over the distal end of the handpiece 2100. The length of the spacer selected is used to determine the distance between the distal end of the handpiece 2100 and the distal end of the spacer 2102 when the spacer is positioned on the handpiece 2100. The variable configuration of the spacer 2102 relative to the distal end of the handpiece 2100 thus enables the user to select a desired device-tissue gap appropriate to the type of treatment being delivered.

Under the treatment approach using this device, the FEDBD handpiece 2100 is positioned adjacent to and over the surface of the implant and NTP or cold plasma and plasma chemistry 2104 is created at the surface of the implant and the implant surface is treated with cold plasma and associated plasma chemistry. In the case of treating a tooth surface prior to the placement of dental material such as bonding a veneer with a resin, cementing a crown, and placement of esthetic resin, the prepared tooth is treated using the FEDBD handpiece for a period of time (e.g., up to 10 minutes, etc.) using the FEDBD handpiece 2100. After treatment of the tooth structure, any biofilm and organic matter is removed and the tooth surface energy and wettability are improved, yielding optimal tooth surface properties to improved dental material performance, e.g., resin bonding.

Regardless of handpiece distal end configuration, the dental handpiece fitted with the spacer 2102/2102 can precisely and effectively deliver NTP plasma and plasma chemistry to surfaces of oral tissues and dental-oral implants (including braces) at the desired device-tissue gap to treat oral tissues and oral devices to disinfect or prepare surfaces for treatment. Further, the FEDBD NTP dental handpiece is configured for use in the treatment of pre-malignant and malignant oral lesions. Additionally, other benign conditions can be treated such as periodontal disease, oral candidiasis, and oral mucositis.

### Controlling the Ambient Atmosphere Gas Mixture in the Delivery of NTP Using a FEDBD

FIG. 22 shows components of an NTP system or device 2200, under an embodiment. The NTP device 2200 of this example comprises a control unit 2201 that delivers energy to the FEDBD handpiece 2202 via an electrical coupling or connection 2206. Using the control unit 2204, the user can change the electrical parameters of the control unit, which in turn varies the electric field of the FEDBD handpiece 2202, yielding the desired plasma and associated plasma chemistry to be delivered at the treatment site. NTP 2208 is formed by the electric field created between the distal end of the handpiece 2202 and the treatment target tissue 2210 by the applied electric field, which ionizes the surrounding ambient air, creating plasma cocktail consisting of reactive oxygen species, metastable species, ozone, electrons, ultraviolet radiation, reactive nitrogen species, positive ions, negative ions, free radicals, and electromechanical effects, as described in detail herein. FIG. 23 shows components of an NTP system 2300 including a controllable environment surrounding the distal end of the FEDBD handpiece 2302, under an alternative embodiment. The controllable environment is generated by varying the ambient atmosphere surrounding the distal end of the handpiece by delivering and varying mixtures of gases (oxygen, nitrogen, etc.) to the distal end of the FEDBD handpiece 2302 for generation of plasma 2330. A plastic sleeve 2304 can be fitted over the distal end of the FEDBD handpiece 2302. The plastic sleeve houses a number of conduits for different functional purposes. For example, a conduit bonded tubing 2306 coupled or connected to a source of gas or gasses 2310 can deliver a gas or gas mixture to the distal end of the FEDBD handpiece 2302 and near the target tissue. A second conduit with tubing 2308 coupled or connected to a suction device 2312 is configured to remove gas mixture from the treatment site. The suction conduit 2308 can also be configured to remove fluids from the treatment site. As a further example, a third conduit (not shown) can be configured to provide a working channel for an endoscopy to view the distal end of the FEDBD handpiece 2302 and the target site 2320.

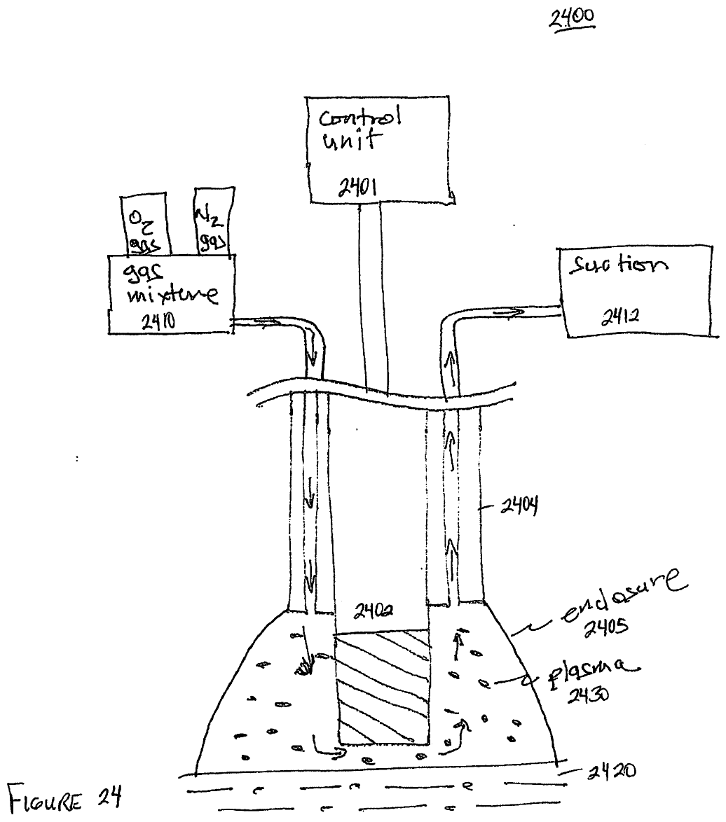

FIG. 24 shows components of an NTP system 2400 including a controllable environment surrounding the distal end of the FEDBD handpiece 2402 and an enclosure coupled to the distal end of a sleeve 2404 surrounding the distal end of the FEDBD handpiece 2402, under another alternative embodiment. The sleeve 2404 is configured to encompass or surround the treatment site 2420 to which the gas or gas mixture is delivered but is not so limited. The enclosure comprises a clear molded plastic piece that can be bonded to the plastic sleeve, but alternative embodiments of the enclosure can comprise any number of alternative materials. The gas mixture can be humidified or dehumidified, and the enclosure provides a more controlled environment to deliver a desired gas or mixture of gas, with or without humidification.

In addition to the dental applications described herein, the NTP device configurations of embodiments described herein can be used to treat a number of medical conditions including but not limited to benign, premalignant, and malignant skin lesions as well as oral, vaginal, and rectal lesions, and toenails with fungal infections can also be treated.

### Treatment of Oral Tissues Using NTP

Intraoral device delivery for the treatment of oral and dental issues using cold plasma and associated plasma chemistry often involves special design considerations secondary to access to and the anatomy of various intraoral structures. Embodiments herein therefore focus on the treatment of oral lesions and dental conditions. For example, surgical tonsillar resection and post-resection treatment of resected tumor site. In the case of implant revision procedures, the delivery of cold plasma and plasma chemistry using the FEDBD handpiece coupled to control unit, wherein cold plasma and plasma chemistry removes biofilm from the dental implant surface and reduces the bacterial load, improving the clinical outcomes of dental implant revision procedures. Similarly, prepared tooth surfaces to receive dental materials or cementation of a dental prosthetic can be pre-treated using cold plasma and associated plasma chemistry to remove organics and biofilm from the prepared surface of both the tooth structure and surface of the dental prosthetic that interfaces with tooth structure to be bonded or cemented.

The embodiments described herein include numerous configurations of an FEDBD dental handpiece, wherein the distal end of the handpiece is cylindrical in shape and can be optionally fitted with a disposable polymer spacer reversibly coupled to the distal end of the handpiece as described in detail herein. In the latter, the dental handpiece can precisely and effectively deliver cold plasma and plasma chemistry to surfaces of oral tissues and dental-oral implants (including braces) at the desired device-tissue gap to treat oral tissues and oral devices to disinfect or prepare surfaces for treatment.

The embodiments herein can be used to treat prepared tooth surfaces as well as to treat benign, pre-malignant and malignant oral lesions. In addition, other benign conditions can be treated such as periodontal disease, oral candidiasis, oral mucositis, and other conditions such as geographic tongue.

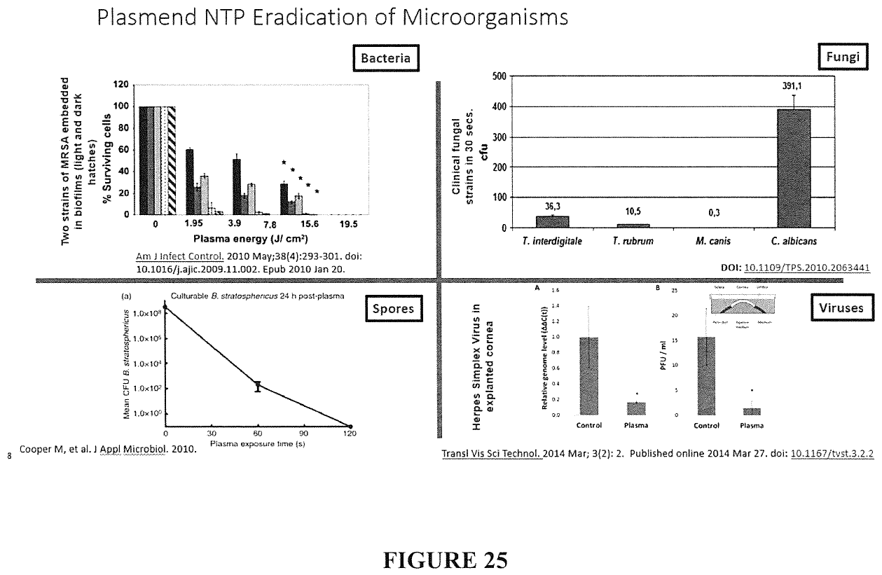

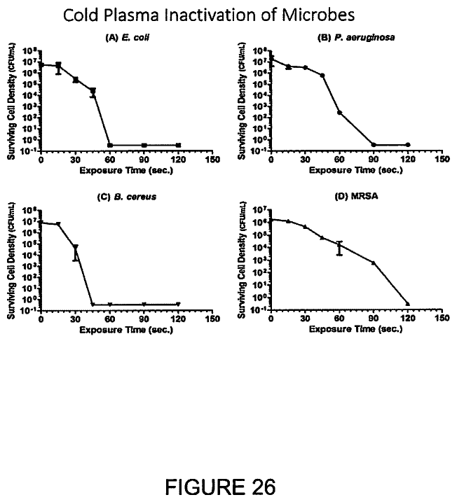

FIG. 25 includes tables showing effectiveness of NTP in eradicating microorganisms, under an embodiment. FIG. 26 includes tables showing effectiveness of cold plasma in the inactivation of microbes, under an embodiment.

### NTP Onco-Immunotherapy

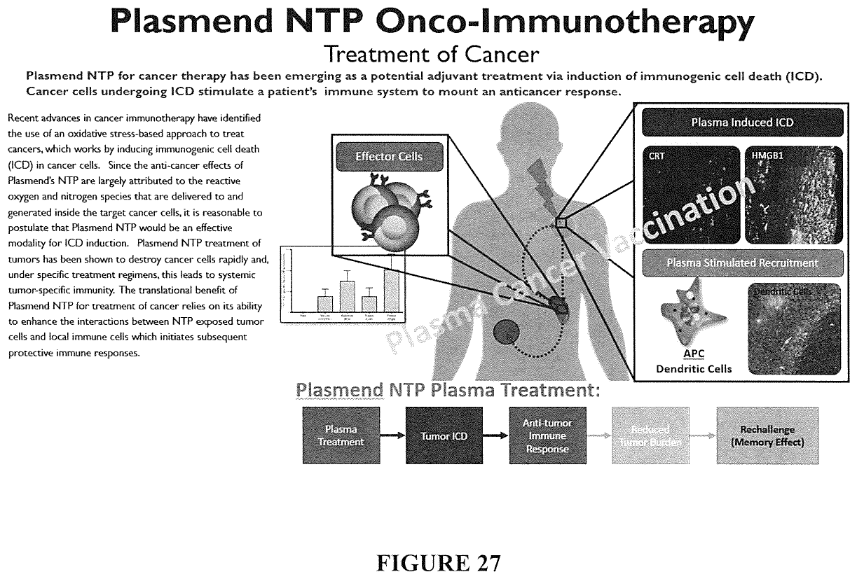

Recent advances in cancer immunotherapy have identified the use of an oxidative stress-based approach to treat cancers, which works by inducing immunogenic cell death (ICD) in cancer cells. Since the anti-cancer effects of NTP are largely attributed to the reactive oxygen and reactive nitrogen species that are delivered to and generated inside the target cancer cells, it is reasonable to postulate that NTP would be an effective modality for ICD induction. NTP treatment of tumors has been shown to destroy cancer cells rapidly and, under specific treatment regimens, this leads to systemic tumor-specific immunity. The translational benefit of NTP for treatment of cancer relies on its ability to enhance the interactions between NTP exposed tumor cells and local immune cells which initiates subsequent protective immune responses. NTP clinical devices described herein along with their corresponding treatment protocols can become an essential part of the therapeutic armament against cancer. FIG. 27 is a block diagram showing NTP-based onco-immunotherapy, under an embodiment.

### Treatment of Cervical Premalignant Cells Using Cold Plasma

Precancerous or premalignant conditions of the cervix involve changes to cervical cells that make them more likely to develop into a malignancy or cancer. Many clinical presentations of changes in cervical cells serve as a basis to remove these abnormal cells via surgery, cryosurgery, laser ablation therapy, or other methods. The NTP devices described herein however can be used to treat lesions in a cavity whether naturally occurring or surgically formed.

Advances in cancer immunotherapy have identified the use of an oxidative stress-based approach to treat cancers, which works by inducing immunogenic cell death (ICD) in cancer cells. Since the anti-cancer effects of NTP are largely attributed to the reactive oxygen and reactive nitrogen species that are delivered to and generated inside the target cancer cells, it is reasonable that NTP would be an effective modality for ICD induction. NTP treatment of tumors has been shown to destroy cancer cells rapidly and, under specific treatment regimens, this leads to systemic tumor-specific immunity. The translational benefit of NTP for treatment of cancer relies on its ability to enhance the interactions between NTP exposed tumor cells and local immune cells which initiates subsequent protective immune responses. The NTP clinical devices and treatment protocols of embodiments described herein can become an essential part of the therapeutic armament against cancer.

Embodiments described herein comprise devices configured to be placed adjacent to or fitted over the cervix to deliver FEDBD cold plasma and plasma chemistry over the surface of the cervix. An FEDBD handpiece with an appropriately sized distal tip can be maneuvered over the cervix during treatment.

### Treatment of Barrett's Esophagus, Prostate Cancer, and Intestinal Conditions Using Side-Firing Cold Plasma

Barrett's esophagus is a potentially serious complication of gastroesophageal reflux disease (GERD). In Barrett's esophagus, normal tissue lining the esophagus changes to tissue that resembles the lining of the intestine. Approximately ten percent of people with chronic symptoms of GERD develop Barrett's esophagus. Approximately one percent of people with Barrett's esophagus develop this particular cancer.

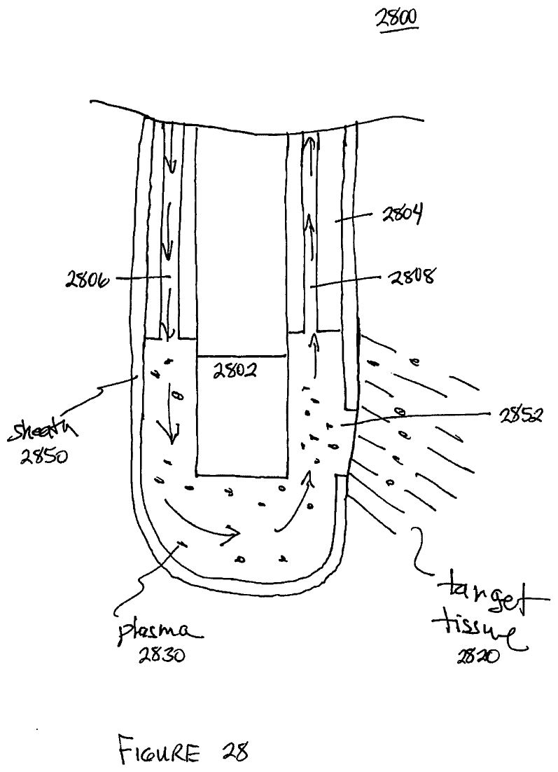

FIG. 28 shows an NTP system 2800 comprising a side-firing cold plasma configuration, under an embodiment. The side-firing configuration of this device 2800 embodiment enables NTP delivery in a duct or similar anatomical structure (e.g., esophagus, urethra-bladder-ureter, colon-intestine, etc.). This device 2800 comprises an NTP system including a controllable environment 2830 surrounding the distal end of the FEDBD handpiece 2802 (for example, see FIG. 23) as described herein. The controllable environment 2830 is generated by varying the ambient atmosphere surrounding the distal end of the handpiece by delivering and varying mixtures of gases (oxygen, nitrogen, etc.) to the distal end of the FEDBD handpiece 2802 for generation of plasma 2830. A plastic sleeve 2804 can be fitted over the distal end of the FEDBD handpiece 2802. The plastic sleeve 2804 houses a number of conduits for different functional purposes. For example, a conduit bonded tubing 2806 coupled or connected to a source of gas or gasses can deliver a gas or gas mixture to the distal end of the FEDBD handpiece 2802 and near the target tissue 2820. A second conduit with tubing 2808 coupled or connected to a suction device is configured to remove gas mixture from the treatment site. The suction conduit 2308 can also be configured to remove fluids from the treatment site.

The FEDBD handpiece 2802 is further fitted in a polymer tubing or sheath 2850 wherein the inside diameter of the sheath can receive the FEDBD handpiece 2802 and the sleeve 2804. The sheath 2850 of an embodiment is round on the distal end with a window 2852 positioned on the sheath wall but is not so limited. This window 2852 causes the plasma and plasma chemistry to be delivered from the side of the sheath thereby enabling the side-firing configuration of the device. The window 2852 allows the cold plasma and plasma chemistry to be delivered to the treatment site with the side wall placed adjacent to the treatment site and cold plasma and plasma chemistry delivered to the target site via the window.

The side-firing configuration of the NTP system 2800 enables the FEDBD handpiece 2802 to be placed under direct vision or endoscopic delivery. In the latter, the working channel of an endoscope can be used to deliver the sheathed NTP device to a target site, e.g., gastroesophageal junction to treat Barrett's esophagus, via the urethra in the area of the prostate, or to a site in the colon or intestine. Optionally, to assist with stability and placement in a duct, the system 2800 can include an inflatable balloon placed distally, laterally, or proximal to the distal end of the sheath-tubing. In other optional configurations, the system 2800 can include another lumen or conduit configured to place an endoscopy or other visualization technology to assist with guidance of the device to the treatment site.

### Delivery of End-Firing Cold Plasma in Laparoscopic and Open Surgical Procedures.

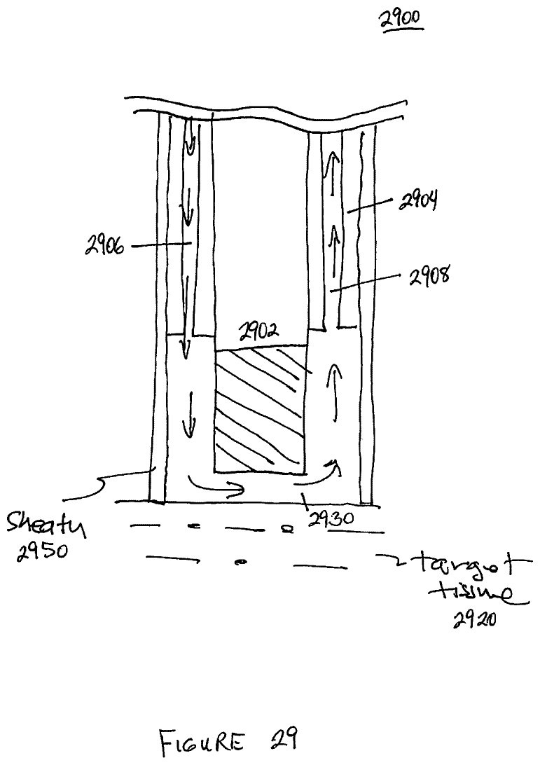

FIG. 29 shows an NTP system 2900 comprising an end-firing cold plasma configuration, under an embodiment. The end-firing configuration of this device embodiment 2900 enables NTP delivery to target tissue under laparoscopic guidance via a trocar or working channel in the laparoscope or under direct vision in open surgical procedures. This device 2900 comprises an NTP system including a controllable environment 2930 at the distal end of the FEDBD handpiece 2902 (see e.g., FIG. 23) as described herein. The controllable environment 2930 is generated by varying the ambient atmosphere surrounding the distal end of the handpiece by delivering and varying mixtures of gases (oxygen, nitrogen, etc.) to the distal end of the FEDBD handpiece 2902 for generation of plasma 2930. A plastic sleeve 2904 is fitted over the distal end of the FEDBD handpiece 2902. The plastic sleeve 2904 houses a number of conduits for different functional purposes. For example, a conduit bonded tubing 2906 coupled or connected to a source of gas or gasses can deliver a gas or gas mixture to the distal end of the FEDBD handpiece 2902 and near the target tissue 2920. A second conduit with tubing 2908 coupled or connected to a suction device is configured to remove gas mixture from the treatment site. The suction conduit 2908 can also be configured to remove fluids from the treatment site for example.

The FEDBD handpiece 2902 is further fitted in a polymer tubing or sheath 2950 wherein the inside diameter of the sheath can receive the FEDBD handpiece 2902 and the sleeve 2904. The sheath 2950 of an embodiment is open on the distal end. The distance from the distal tip of the FEDBD handpiece 2902 to the end of the most distal aspect of open-ended sheath can be varied. This open-ended sheath enables plasma and plasma chemistry 2930 to be delivered to the treatment site 2920 when the distal end of the sheath 2950 is placed over the target tissue.

The FEDBD handpiece 2902 of the NTP system 2900 can be placed under direct vision or laparoscopic-endoscopic delivery. In the latter, the working channel of a laparoscope endoscope can be used to deliver the sheathed NTP device to a target site, treatment of cancers in the abdomen. Alternatively, the FEDBD handpiece 2902 can be delivered via a trocar to the treatment site and under laparoscopic-endoscopic guidance.

### Treatment of Surgically Resected Tumor Sites using NTP Technology

Glioblastoma, breast cancer, and colorectal cancers are examples of the use of NTP technology in the surgical excision of cancer and treatment of the surgical bed. For example, glioblastoma is surgically resected under stereotactic surgical techniques. Post-resection, the FEDBD handpiece described herein can be secured in the stereotactic frame and placed into the resected tumor site. The FEDBD handpiece is energized and cold plasma and plasma chemistry is delivered to the tumor resection bed, treating tissues of the resected bed including cancer cells that have not been removed. It is believed, for example, that cytomegalovirus (CMV) may be an etiologic factor in the formation of glioblastoma and, as such, exposure of glioblastoma cells with CMV to FEDBD NTP and plasma chemistry may illicit an ICD-vaccinated approach to the treatment of glioblastoma. In the resection of breast cancer, the surgical bed can be treated using the device embodiments described herein (see e.g., FIGS. 23, 24, 28, and 29).

Colorectal cancer is often surgically excised using laparoscopic assisted surgery. Post-resection, an FEDBD handpiece can be placed into the resected colorectal tumor bed via a trocar and under laparoscopic video guidance. The surgeon can select the gas or gas mixture to be delivered to the target sit via the device embodiments described herein (see e.g., FIGS. 28 and 29).

Embodiments comprise an FEDBD handpiece coupled to a powered control to create and deliver cold plasma and plasma chemistry over resected tumor sites. The device is configured to expose the surgical margins of a resected tumor site to cold plasma and plasma chemistry, and to expose the tumor to cold plasma and plasma chemistry.

The FEDBD device of embodiments herein is configured for open surgical, endoscopic, and other minimally invasive surgical techniques, as well as stereotactic and other guidance techniques.

### Indirect Treatment of Tissue Using FEDBD NTP

The FEDBD device of embodiments described herein is configured to deliver cold plasma and plasma chemistry directly to fluids that can then be delivered to the body.

Embodiments comprise use of the FEDBD device in a batch process or continuous process. The FEDBD device is also configured to monitor the subject fluid for pH and hydrogen peroxide content. After the fluid is exposed to cold plasma and plasma chemistry, it can be delivered to the body topically, as lavage during surgery, or injected in a tissue or tumor.

Embodiments include a system comprising a non-thermal plasma (NTP) device including a handpiece configured to generate NTP at a treatment site comprising an intraoral region. A sleeve is configured to removably couple to a distal region of the handpiece. Embodiments include a system comprising: a non-thermal plasma (NTP) device comprising a handpiece configured to generate NTP at a treatment site comprising an intraoral region; and a sleeve configured to removably couple to a distal region of the handpiece.

The distal region of the handpiece comprises a cylindrical-shaped configuration.

The sleeve is configured to slide over the distal region of the handpiece and includes a cradle coupled to a distal end of the sleeve.

When the sleeve is fitted over the distal end of the handpiece, a distance between the cradle and the distal end of the handpiece is variable, wherein the distance controls selection of a device-tissue gap appropriate to a treatment type.

The distal region of the handpiece comprises a pencil-shaped configuration.

The sleeve is configured to slide over the distal region of the handpiece and includes an open distal end.

When the sleeve is fitted over the distal end of the handpiece, a distance between a distal end of the sleeve and the distal end of the handpiece is variable, wherein the distance controls selection of a device-tissue gap appropriate to a treatment type.

The sleeve is configured to slide over the distal region of the handpiece and includes an open distal end through which the distal end of the handpiece protrudes, wherein the distal region of the handpiece includes the distal end of the handpiece.

The sleeve includes a first conduit configured to couple to a gas source, wherein the first conduit is configured to deliver a gas to a region adjacent to the distal end of the handpiece and treatment site.

The sleeve includes a second conduit configured to couple to a vacuum source, wherein the second conduit is configured to remove at least one of fluids and a mixture including the gas from a region adjacent to the distal end of the handpiece and treatment site.

The sleeve includes a third conduit configured as an instrument port configured to receive a medical instrument directed to the region adjacent to the distal end of the handpiece and treatment site.

The third conduit is configured to receive an endoscope to view the region adjacent to the distal end of the handpiece and treatment site.

The system comprises an enclosure coupled to a distal region of the sleeve and configured to surround the distal end of the handpiece and treatment site to provide a controlled treatment environment.

The system comprises a sheath coupled to an outer region of the sleeve and enclosing the sleeve and the distal end of the handpiece.

The sheath includes a window in a side wall, wherein the window is configured to direct side-firing cold plasma toward the treatment site.

The sheath includes an open distal end configured to direct end-firing cold plasma toward the treatment site.

The NTP device comprises a floating electrode dielectric barrier discharge (FEDBD) device.

The system comprises a control unit coupled to the NTP device, wherein the control unit controls the plasma and associated plasma chemistry delivered at the treatment site via control of an electric field of the FEDBD device.

Embodiments include a system comprising a floating electrode dielectric barrier discharge (FEDBD) device configured to generate non-thermal plasma (NTP) at a treatment site comprising an intraoral region. A sleeve is configured to slide over the distal region of the FEDBD device and comprising an open distal end through which a distal end of the FEDBD device protrudes. The sleeve is configured to control delivery of the NTP to the treatment site.

Embodiments include a system comprising: a floating electrode dielectric barrier discharge (FEDBD) device configured to generate non-thermal plasma (NTP) at a treatment site comprising an intraoral region; and a sleeve configured to slide over the distal region of the FEDBD device and comprising an open distal end through which a distal end of the FEDBD device protrudes, wherein the sleeve is configured to control delivery of the NTP to the treatment site.

It should be observed that the embodiments described in detail herein in accordance with the present invention reside primarily in combinations of method steps and apparatus components related to a cold plasma therapy device used with supersaturated gas emulsion (SSGE). Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims.

What is claimed is:

1. A system comprising:
a non-thermal plasma (NTP) device comprising a handpiece configured to generate NTP at a treatment site comprising an intraoral region; and
a sleeve configured to removably couple to a distal region of the handpiece.

2. The system of claim 1, wherein the distal region of the handpiece comprises a cylindrical-shaped configuration.

3. The system of claim 2, wherein the sleeve is configured to slide over the distal region of the handpiece and includes a cradle coupled to a distal end of the sleeve.

4. The system of claim 3, wherein, when the sleeve is fitted over the distal end of the handpiece, a distance between the cradle and the distal end of the handpiece is variable, wherein the distance controls selection of a device-tissue gap appropriate to a treatment type.

5. The system of claim 1, wherein the distal region of the handpiece comprises a pencil-shaped configuration.

6. The system of claim 5, wherein the sleeve is configured to slide over the distal region of the handpiece and includes an open distal end.

7. The system of claim 6, wherein, when the sleeve is fitted over the distal end of the handpiece, a distance between a distal end of the sleeve and the distal end of the handpiece is variable, wherein the distance controls selection of a device-tissue gap appropriate to a treatment type.

8. The system of claim 1, wherein the sleeve is configured to slide over the distal region of the handpiece and includes an open distal end through which the distal end of the handpiece protrudes, wherein the distal region of the handpiece includes the distal end of the handpiece.

9. The system of claim 8, wherein the sleeve includes a first conduit configured to couple to a gas source, wherein the first conduit is configured to deliver a gas to a region adjacent to the distal end of the handpiece and treatment site.

10. The system of claim 9, wherein the sleeve includes a second conduit configured to couple to a vacuum source, wherein the second conduit is configured to remove at least one of fluids and a mixture including the gas from a region adjacent to the distal end of the handpiece and treatment site.

11. The system of claim 10, wherein the sleeve includes a third conduit configured as an instrument port configured to receive a medical instrument directed to the region adjacent to the distal end of the handpiece and treatment site.

12. The system of claim 11, wherein the third conduit is configured to receive an endoscope to view the region adjacent to the distal end of the handpiece and treatment site.

13. The system of claim 8, comprising an enclosure coupled to a distal region of the sleeve and configured to surround the distal end of the handpiece and treatment site to provide a controlled treatment environment.

14. The system of claim 8, comprising a sheath coupled to an outer region of the sleeve and enclosing the sleeve and the distal end of the handpiece.

15. The system of claim 14, wherein the sheath includes a window in a side wall, wherein the window is configured to direct side-firing cold plasma toward the treatment site.

16. The system of claim 14, wherein the sheath includes an open distal end configured to direct end-firing cold plasma toward the treatment site.

17. The system of claim 1, wherein the NTP device comprises a floating electrode dielectric barrier discharge (FEDBD) device.

18. The system of claim 17, comprising a control unit coupled to the NTP device, wherein the control unit controls the plasma and associated plasma chemistry delivered at the treatment site via control of an electric field of the FEDBD device.

19. A system comprising:
a floating electrode dielectric barrier discharge (FEDBD) device configured to generate non-thermal plasma (NTP) at a treatment site comprising an intraoral region; and
a sleeve configured to slide over the distal region of the FEDBD device and comprising an open distal end through which a distal end of the FEDBD device protrudes, wherein the sleeve is configured to control delivery of the NTP to the treatment site.
